SEQUENCE LISTING (22 sequences)

SEQ ID NO: 1 - protein, 209 aa, Artificial Sequence [sequence omitted]

SEQ ID NO: 2 - protein, 28 aa, Artificial Sequence
  CTP1
SSSSKAPPPSLPSPSRLPGPSDTPILPQ

SEQ ID NO: 3 - protein, 227 aa, Artificial Sequence
  Amino acid sequence of vFc gamma 1
DKTHTCPPCPAPEVAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 223 aa, Artificial Sequence
  Amino acid sequence of vFc gamma 2-1
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 223 aa, Artificial Sequence
  Amino acid sequence of vFc gamma 2-2
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 6 - protein, 223 aa, Artificial Sequence
  Amino acid sequence of vFc gamma 2-3
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 7 - protein, 229 aa, Artificial Sequence
  Amino acid sequence of vFc gamma 4
ESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 459 aa, Artificial Sequence
  Amino acid sequence of FGF21 fusion protein
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYASGSGGGGSGGGGSGGGGSGGGGSGGGGSSSSSKAPPPSLPSPSRLPGPSDTPILPQVECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 9 - DNA, 1380 bp, Artificial Sequence
  Nucleotide sequence of FGF21 fusion protein
caccctattcccgatagctcccccctcctgcagttcggaggccaggtgaggcagcggtacctgtacaccgacgacgctcagcagaccgaagctcacctggagatcagggaggatggaaccgtcggcggagctgctgaccagtcccccgagagcctgctgcagctgaaggccctgaagcccggagtcatccagatcctgggcgtgaagacctcccggtttctgtgtcagcggcccgatggagccctgtacggctccctgcattttgaccccgaggcctgtagcttcagggagctgctgctggaagacggctacaacgtgtaccagagcgaagctcacggactgcccctgcacctgcctggcaacaaatcccctcacagggaccccgctcccaggggacctgccaggttcctgcctctgcccggactgcctcctgctcctcccgaacctcctggcatcctcgctcctcagccccctgatgtcggcagcagcgaccctctgtccatggtcggccccagccaaggcaggagcccttcctacgcttccggatccggtggcggtggctccggtggaggcggaagcggcggtggaggatcaggcggtggaggtagcggcggaggcggtagctccagctctagtaaagctccccctccttccctgccctcaccctcaagactgcctggaccttccgacactcccatcctgccacaggtggagtgccctccatgtccagcaccccctgtcgcaggtccatctgtgttcctgtttccacccaagcctaaagaccagctgatgatctcccgcaccccagaagtcacctgtgtggtcgtggatgtgagccatgaagaccccgaggtccagttcaattggtacgtggatggcgtcgaggtgcacaacgctaagacaaaacctagagaagagcagttcaactctacctttcgcgtcgtgagtgtgctgacagtcgtgcaccaggactggctgaatggcaaggagtataagtgcaaagtgagcaacaaaggactgcctgcctcaatcgaaaagactatttccaagaccaaaggacagccaagagagccccaggtgtacaccctgcctccaagccgcgaagagatgactaaaaatcaggtctctctgacctgtctggtgaaggggttttatcctagtgatatcgccgtggaatgggagtcaaacggtcagccagagaacaattacaagaccacaccccctatgctggacagcgatgggtctttctttctgtatagcaaactgacagtggacaagtctcggtggcagcagggtaacgtcttctcttgcagtgtgctgcacgaagcactgcacaatcattacacccagaagtcactgtcactgagcccaggaaaatga

SEQ ID NO: 10 - protein, 14 aa, Artificial Sequence
  linker sequence
  MISC_FEATURE (1)..(2): May or may not be present
  MISC_FEATURE (3)..(5): May or may not be present
  MISC_FEATURE (6)..(9): May or may not be present
  MISC_FEATURE (10)..(14): May or may not be present
  See specification as filed for detailed description of substitutions and preferred embodiments
GSGGSGGGSGGGGS

SEQ ID NO: 11 - protein, 5 aa, Artificial Sequence
  L1
GGGGS

SEQ ID NO: 12 - protein, 16 aa, Artificial Sequence
  L2
GSGGGSGGGGSGGGGS

SEQ ID NO: 13 - protein, 27 aa, Artificial Sequence
  L3
GSGGGGSGGGGSGGGGSGGGGSGGGGS

SEQ ID NO: 14 - protein, 35 aa, Artificial Sequence
  L4
GGGGSGGGGSGGGGSGGGGSGGGGSGGGGSGGGGS

SEQ ID NO: 15 - protein, 20 aa, Artificial Sequence
  L5
GGGSGGGSGGGSGGGSGGGS

SEQ ID NO: 16 - protein, 12 aa, Artificial Sequence
  L6
GGSGGSGGSGGS

SEQ ID NO: 17 - protein, 10 aa, Artificial Sequence
  CTP3
SSSSKAPPPS

SEQ ID NO: 18 - protein, 14 aa, Artificial Sequence
  CTP4
SRLPGPSDTPILPQ

SEQ ID NO: 19 - protein, 16 aa, Artificial Sequence
  CTP5
SSSSKAPPPSLPSPSR

SEQ ID NO: 20 - protein, 33 aa, Artificial Sequence
  CTP2
PRFQDSSSSKAPPPSLPSPSRLPGPSDTPILPQ

SEQ ID NO: 21 - protein, 30 aa, Artificial Sequence
  (CTP3)3
SSSSKAPPPSSSSSKAPPPSSSSSKAPPPS

SEQ ID NO: 22 - protein, 32 aa, Artificial Sequence
  CTP5-CTP5
SSSSKAPPPSLPSPSRSSSSKAPPPSLPSPSR

(19) United States
(12) Patent Application Publication
(10) Pub. No.: US 2026/0256885 A1
(43) Pub. Date: Sep. 3, 2026

(54) USES OF FGF21 FUSION PROTEINS

(71) Applicant: AMPSOURCE BIOPHARMA SHANGHAI INC., Pudong New Area Shanghai (CN)

(72) Inventors: Qiang Li, Pudong New Area Shanghai (CN); Zhao Dong, Pudong New Area Shanghai (CN); Yuanli Li, Pudong New Area Shanghai (CN); Chi Zhou, Pudong New Area Shanghai (CN)

(73) Assignee: AMPSOURCE BIOPHARMA SHANGHAI INC., Pudong New Area Shanghai (CN)

(21) Appl. No.: 18/877,671

(22) PCT Filed: Jun. 23, 2022
(86) PCT No.: PCT/CN2022/100721

§ 371 Date: Dec. 20, 2024

(51) Int. Cl. A61K 38/18; A61K 38/24; A61K 38/38; A61K 38/40; A61P 1/16

(52) U.S. Cl. CPC A61K 38/1825; A61K 38/24; A61K 38/38; A61K 38/40; A61P 1/16

(57) ABSTRACT

The present disclosure provides human fibroblast growth factor 21 (hFGF21) fusion proteins for treating non-alcoholic fatty liver disease (NAFLD), diabetes, diabetic complications, obesity, dyslipidemia, metabolic syndrome, and/or disorders associated with severe inactivating mutations in the insulin receptor.

### SEQUENCE LISTING

The instant application contains a Sequence Listing, which has been submitted electronically in ASCII format and is hereby incorporated by reference in its entirety. Said ASCII copy, created on Jun. 20, 2022, is named 15079.0007 seq list_ST25 and is 22.1 K bytes in size.

### FIELD OF THE DISCLOSURE

The present disclosure relates to human fibroblast growth factor 21 (hFGF21) fusion proteins for the treatment of non-alcoholic fatty liver disease (NAFLD), diabetes, diabetic complications, obesity, dyslipidemia, metabolic syndrome, and/or disorders associated with severe inactivating mutations in the insulin receptor.

### BACKGROUND OF THE DISCLOSURE

FGF21 is an atypical member of the fibroblast growth factor family and belongs to the FGF endocrine factor subfamily. Mature human FGF21 contains 181 amino acids and is mainly synthesized by the liver, with a small amount formed in white adipose tissue, and heart and skeletal muscles. FGF21 has a significant beneficial metabolic regulation effect on obese rodent and non-human primates. FGF21 regulates blood glucose levels independent of insulin, and does not cause hypoglycemia when administered as a hypoglycemic agent. FGF21 is the only cytokine currently found in the FGF family that does not have mitogenic effects, thereby greatly reducing the risk of side effects in clinical use. Preclinical and clinical research have shown that FGF21 is a potential target for the treatment of NAFLD, e.g., non-alcoholic steatohepatitis (NASH), diabetes, diabetic complications, obesity, dyslipidemia, metabolic syndrome, and/or disorders associated with severe inactivating mutations in the insulin receptor.

NAFLD is a metabolic stress-induced liver injury closely related to insulin resistance and genetic susceptibility, including non-alcoholic simple fatty liver (NAFL), NASH, NASH-induced liver cirrhosis, and hepatocellular carcinoma. NAFLD not only causes liver disease-related disability and death, but also is related to metabolic syndrome (MetS), type 2 diabetes, arteriosclerotic cardiovascular disease, and colorectal tumors [Guidelines for the prevention and treatment of nonalcoholic fatty liver disease (updated in 2018), Journal of Practical Liver Diseases, 21 (02), 30-39]. With the prevalence of obesity, diabetes, and MetS, NAFLD has become the largest chronic liver disease in China and the primary cause of abnormal liver biochemical indicators in health physical examinations [Lee H W and Vincent Wai-Sun Wong, Changing NAFLD Epidemiology in China. Hepatology, 2019, 70 (4)]. In the absence of other clear causes of liver injury, more than a quarter of adult patients with NAFLD can be inferred to have NASH, marked by elevated serum ALT and AST. [Leonardo M D, Byrne M B, and Caldwell M D, et al., Global epidemiology of nonalcoholic fatty liver disease-meta-analytic assessment of prevalence, incidence, and outcomes, Hepatology, 2016; 64 (1): 73-84]. NASH can lead to the progression of liver fibrosis and liver cirrhosis, eventually leading to liver failure and death [Williams C D, Stengel J, and Asike M I, et al., Prevalence of nonalcoholic fatty liver disease and nonalcoholic steatohepatitis among a largely middle-aged population utilizing ultrasound and liver biopsy: a prospective study, Gastroenterology, 2011, 140 (1): 124-131].

There is no therapy approved for the treatment of NASH in the world. New drugs are currently being developed targeting metabolic regulation and inflammatory processes, including farnesoid X receptor (FXR) agonists, peroxisome proliferator-activated receptor (PPAR) agonists, acetyl-CoA carboxylase (ACC) inhibitors, stearoyl-CoA desaturase-1 (SCD-1) inhibitor dual antagonism of cell surface chemokine receptor 2 (CCR2) and cell surface chemokine receptor 5 (CCR5) agents, Apoptosis Signal-Regulated Kinase 1 (ASK1) inhibitors, Caspase inhibitors, Galectin-3 inhibitors, thyroid hormone receptor-beta (THR-beta) agonists, lysyl oxidase-like 2 (LOXL2) monoclonal antibodies, fibroblast growth factor 19 (FGF19) analogs, and fibroblast growth factor 21 (FGF21) analogs, etc.

Although findings in animal models suggest potential therapeutic advantages of human FGF21, its physicochemical properties limit clinical applications. The molecular weight of natural FGF21 is about 20 kDa, which can be readily excreted through glomerular filtration, resulting in a short half-life in vivo. The half-life of FGF21 in primates is 1 to 2h. A small number of amino acid deletions at the C-terminus of native FGF21 can lead to its biological activity significantly reduced [Shanaka S, Randy H, Junming Y, et al., A novel Fc-FGF21 with improved resistance to proteolysis, increased affinity towards β-Klotho and enhanced efficacy in mice and cynomolgus monkeys, Endocrinology, 2017 (5): 5]. The C-terminus of native FGF21 has multiple protease hydrolysis sites, which are easily hydrolyzed by proteases; as a result, native FGF21 has poor in vivo stability and reduced biological activity. At the same time, natural FGF21 is prone to aggregation and has poor stability. Although FGF21 has certain curative effect, the absorption of FGF21 in clinical medication is extremely low and the manufacture process is difficult, which limits the therapeutic use of FGF21.

A variety of long-acting technologies for protein modification have been reported to extend the in vivo half-life of recombinant FGF21, for example, by linking FGF21 to a PEG molecule to increase the molecule weight to reduce the glomerular filtration rate and prolong the in vivo retention time (see WO2005/091944, WO2006/050247, WO2008/121563 and WO2012/066075), infusing FGF21 with a fatty acid long chain which is capable of binding to serum albumin (see WO2010/084169 and WO2012/010553), or providing an agonist antibody that specifically binds to the FGFR or FGFR/B-klotho complex to activate the FGF/FGFR signaling pathway by mimicking the action mechanism of FGF21 (see WO2011/071783, WO2011/130417, WO2012/158704 and WO2012/170438), or infusing with an Fc fragment to improve the half-life of FGF21 (see WO2004/110472, WO2005/113606, WO2009/149171, WO2010/042747, WO2010/129503, WO2010/129600, WO2013/049247, WO2013/188181, and WO2016/114633). Certain FGF21 fusion proteins comprising a wild-type human fibroblast growth factor 21 or an analog thereof, a flexible peptide linker, at least one rigid unit, and a fusion ligand are described in WO 2018/032785.

There are currently no FGF21 analog preparations available for treating NASH indications. Several FGF21 long-acting analogs are in various stages of clinical research: (1) LY2405319, a recombinant human FGF21 analog developed by Eli Lilly that introduced Leu118Cys and Ala134Cys mutations in human FGF21, (2) PF-05231023, a long-acting FGF21 analog developed by Pfizer that introduced cysteines into recombinant human FGF21 through mutations at D79C, H125C, and A129C, wherein the recombinant human FGF21 is fused with an antibody scaffold, (3) BMS-986036, a PEGylated FGF21 analog developed by Bristol-Myers Squibb Company, and (4) AKR-001, an Fc-FGF21 fusion protein that Akero licensed from Amgen where the N-terminus of a recombinant human FGF21 is fused with an IgG Fc fragment and the L98R, P171G, and A180E mutations were introduced at the C-terminus of FGF21.

The present disclosure provides recombinant human FGF21-Fc fusion proteins for the treatment of NAFLD, e.g., NASH, diabetes, diabetic complications, obesity, dyslipidemia, metabolic syndrome, and/or disorders associated with severe inactivating mutations in the insulin receptor.

### SUMMARY OF THE DISCLOSURE

In some embodiments, the present disclosure provides a method of treating a disease or disorder, comprising administering to a subject in need thereof a therapeutically effective amount of an FGF21 fusion protein at least once every four weeks, wherein the therapeutically effective amount of the FGF21 fusion protein is 5 mg or more (e.g., 5 mg to 500 mg), wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the human fibroblast growth factor 21 or an analog thereof is selected from polypeptides having an amino acid sequence at least 95% homologous to that of wild-type hFGF21, the flexible peptide linker is selected from L1, L2, L3, L4, and L5, the at least one rigid unit is selected from CTP1, CTP2, CTP3, CTP4, and CTP5 as disclosed herein, and the fusion partner is selected from vFcγ1, vFcγ2-1, vFcγ2-2, vFcγ2-3, and vFcγ4.

In some embodiments, the present disclosure provides an FGF21 fusion protein or a pharmaceutical composition thereof for use in treating a disease or disorder, wherein a therapeutically effective amount of an FGF21 fusion protein is administered to a subject in need thereof at least once every four weeks, wherein the therapeutically effective amount of the FGF21 fusion protein is 5 mg or more (e.g., 5 mg to 500 mg), wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the present disclosure provides use of an FGF21 fusion protein or a pharmaceutical composition thereof in the manufacture of a medicament for treating a disease or disorder, wherein a therapeutically effective amount of an FGF21 fusion protein is administered to a subject in need thereof at least once every four weeks, wherein the therapeutically effective amount of the FGF21 fusion protein is 5 mg or more (e.g., 5 mg to 500 mg), wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the FGF21 fusion protein comprises an amino acid sequence of SEQ ID NO. 8.

In some embodiments, the therapeutically effective amount of the FGF21 fusion protein is from 5 mg to 400 mg.

In some embodiments, the therapeutically effective amount of the FGF21 fusion protein is from 10 mg to 300 mg.

In some embodiments, the therapeutically effective amount of the FGF21 fusion protein is from 15 mg to 60 mg.

In some embodiments, the therapeutically effective amount of the FGF21 fusion protein is from 15 mg to 37.5 mg.

In some embodiments, the therapeutically effective amount of the FGF21 fusion protein is administered at least once every three weeks, at least once every two weeks, or at least once every week.

### DETAILED DESCRIPTION OF THE DISCLOSURE

All publications, including but not limited to patents and patent applications, cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

### Definitions

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice of the present disclosure, exemplary materials and methods are described herein.

As used in this specification and the appended claims, the singular forms “a,” “an,” and “the” include plural referents unless the content clearly dictates otherwise. Thus, for example, reference to “a protein” includes one, two, or more proteins, and the like.

As used herein, the term “about” will be understood by persons of ordinary skill in the art and will vary to some extent depending on the context in which it is used. In some embodiments, the term “about” when referring to a measurable value such as an amount, a temporal duration, and the like, is meant to encompass art-accepted variations based on standard errors in making such measurements. In some embodiments, the term “about” when referring to such values, is meant to encompass variations of ±20% or ±10%, more preferably ±5%, even more preferably ±1%, and still more preferably ±0.1% from the specified value, as such variations are appropriate to perform the disclosed methods.

Unless indicated otherwise, all numerical ranges are inclusive of the values defining the range.

As used herein, the term “range,” “ranges,” or “ranging” from a scope means that the value at issue is equal or higher than the minimum value of the scope provided, and is equal or lower than the maximum value of the scope provided. The term “from X to Y” means a range from X to Y, including the values of X and Y and the values in between X and Y.

The term “human FGF21” or “hFGF21” as used in the present disclosure refers to a wild-type human FGF21 polypeptide as well as an analog thereof. As used herein, the term “wild-type” or “native” means naturally occurring.

The term “FGF21 fusion protein” as used in the present disclosure refers to a protein, in the order from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion ligand (such as an immunoglobulin, an Fc fragment thereof, an albumin, or transferrin). The term “FGF21-Fc fusion protein” or “FGF21-Fc” as used herein refers to an FGF21 fusion protein wherein the fusion ligand is an Fc.

As used herein, a “composition” refers to any mixture of two or more products, substances, or compounds, including but not limited to, proteins, antibodies, polynucleotides, vectors, or cells. It may be a solution, a suspension, liquid, powder, a paste, aqueous, non-aqueous, or any combination thereof.

As used herein, a “pharmaceutical composition” refers to an active pharmaceutical agent formulated in pharmaceutically acceptable or physiologically acceptable forms for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. Some non-limiting examples of the components that could be included in the composition are carriers, stabilizers, diluents, dispersing agents, suspending agents, thickening agents, and/or excipients. The pharmaceutical composition facilitates administration of the FGF21 fusion protein described herein to a subject.

Multiple techniques of administration of a pharmaceutical composition exist in the art, including, but not limited to, parenteral, oral, aerosol, parenteral, ophthalmic, pulmonary, and topical administration. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. Parenteral administration can be by continuous infusion over a selected period of time.

As used herein, the term “pharmaceutically acceptable” refers to a material, such as a carrier or diluent, which does not abrogate the biological activity or properties of a therapeutic substance, and is relatively nontoxic, i.e., the material may be administered to a subject without causing undesirable biological effects or interacting in a deleterious manner with any of the components of the composition in which it is contained. Pharmaceutically acceptable components include those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “therapeutically effective amount” refers to an amount of an active pharmaceutical agent or pharmaceutical composition that is sufficient to significantly and positively modify the symptoms and/or conditions to be treated (e.g., provide a positive clinical response). The effective amount of an active pharmaceutical agent or pharmaceutical composition will vary with the particular condition being treated, the severity of the condition, the duration of treatment, the nature of concurrent therapy, the particular composition being employed, the particular pharmaceutically-acceptable excipient(s) and/or carrier(s) utilized, and like factors with the knowledge and expertise of the attending physician.

As used herein, a “disease” or “disorder” refers to a condition in which treatment is needed and/or desired.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

As used herein, the terms “individual” and “subject” are used interchangeably herein to refer to an animal. For example, in some embodiments, the animal is a mammal. The animal can be male or female and can be at any suitable age, including infant, juvenile, adolescent, adult, and geriatric. In some examples, an “individual” or “subject” refers to an animal in need of treatment for a disease or disorder. In some embodiments, the animal to receive the treatment can be a “patient,” designating the fact that the animal has been identified as having a disorder of relevance to the treatment, or being at adequate risk of contracting the disorder. In particular embodiments, the animal is a human, such as a human patient.

The term “disorders associated with severe inactivating mutations in the insulin receptor” describes conditions in subjects with mutations in the insulin receptor (or possible proteins directly downstream from it) which cause severe insulin resistance but often occurring without obesity commonly found in type 2 diabetes. In many ways, subjects afflicted with these conditions manifest hybrid symptoms of type 1 diabetes and Type 2 diabetes. Subjects thereby afflicted fall into several categories of roughly increasing severity, including Type A Insulin Resistance, Type C Insulin Resistance (AKA HAIR-AN Syndrome), Rabson-Mendenhall Syndrome, and Donohue's Syndrome or Leprechaunism. These disorders are associated with very high endogenous insulin levels, resulting in hyperglycemia. Subjects thereby afflicted also exhibit various clinical features associated with “insulin toxicity,” including hyperandrogenism, polycystic ovarian syndrome (PCOS), hirsutism, and acanthosis nigricans (excessive growth and pigmentation in the folds of the skin).

“Diabetic complications” are dysfunctions in subjects caused by chronic hyperglycemia such as diabetic nephropathy, diabetic neuropathy, diabetic feet (foot ulcers and poor circulation), and ocular lesions (retinopathies). Diabetes also increases the risk for heart diseases and bone and joint disorders. Other long-term complications of diabetes include skin problems, digestive problems, sexual dysfunction, and problems with teeth and gums.

“Metabolic syndrome (MS)” is a pathological condition in which a variety of metabolic components are abnormally aggregated, including: (1) abdominal obesity or overweight; (2) atherosclerosis and dyslipidemia, such as hypertriglyceridemia (TG) and low high-density lipoprotein cholesterol (HDL-C); (3) hypertension; (4) insulin resistance and/or increased pro-inflammatory state (C-reactive protein, CRP), and increased pro-thrombotic state (increased fibrinogen and plasminogen inhibitor-1, PAI-1).

“Dyslipidemia” refers to a disorder of lipoprotein metabolism, including lipoprotein overproduction or deficiency. Dyslipidemias may be manifested by an increase in the total cholesterol, low-density lipoprotein (LDL) cholesterol and triglyceride concentrations, and a decrease in high-density lipoprotein (HDL) cholesterol concentration in the blood.

“Non-alcoholic fatty liver disease (NAFLD)” is a spectrum of liver disease characterized by hepatic steatosis, in the absence of excessive alcohol consumption.

“Non-alcoholic steatohepatitis (NASH)” is a spectrum of liver disease that includes simple steatosis, lobular inflammation, and/or progressing to fibrosis, in the absence of excessive alcohol consumption.

### Methods of Treatment and Uses

In some embodiments, the present disclosure provides a method of treating a disease or disorder, comprising administering to a subject in need thereof, a therapeutically effective amount of an FGF21 fusion protein or a pharmaceutical composition comprising an FGF21 fusion protein, wherein the disease or condition includes, but is not limited to, non-alcoholic fatty liver disease (NAFLD), diabetes, diabetic complications, obesity, dyslipidemia, metabolic syndrome, and/or disorders associated with severe inactivating mutations in the insulin receptor.

In some embodiments, disclosed herein is an FGF21 fusion protein or a pharmaceutical composition comprising an FGF21 fusion protein, for use as a medicament.

In some embodiments, disclosed herein is use of an FGF21 fusion protein or a pharmaceutical composition comprising an FGF21 fusion protein, for the manufacture of a medicament for treating a disease or condition includes, but is not limited to, non-alcoholic fatty liver disease (NAFLD), diabetes, diabetic complications, obesity, dyslipidemia, metabolic syndrome, and/or disorders associated with severe inactivating mutations in the insulin receptor

In some embodiments, the present disclosure provides an FGF21 fusion protein or a pharmaceutical composition comprising an FGF21 fusion protein for use in treating a disease or condition includes, but is not limited to, non-alcoholic fatty liver disease (NAFLD), diabetes, diabetic complications, obesity, dyslipidemia, metabolic syndrome, and/or disorders associated with severe inactivating mutations in the insulin receptor.

In some embodiments, the present disclosure provides a method of treating a disease or disorder, comprising administering to a subject in need thereof a therapeutically effective amount of an FGF21 fusion protein at least once every four weeks, wherein the therapeutically effective amount of the FGF21 fusion protein is 5 mg or more, wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the present disclosure provides an FGF21 fusion protein or a pharmaceutical composition thereof for use in treating a disease or disorder wherein a therapeutically effective amount of an FGF21 fusion protein is administered to a subject in need thereof at least once every four weeks, wherein the therapeutically effective amount of the FGF21 fusion protein is 5 mg or more, wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the present disclosure provides use of an FGF21 fusion protein or a pharmaceutical composition thereof in the manufacture of a medicament for treating a disease or disorder wherein a therapeutically effective amount of an FGF21 fusion protein is administered to a subject in need thereof at least once every four weeks, wherein the therapeutically effective amount of the FGF21 fusion protein is 5 mg or more, wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

In some embodiments, the human fibroblast growth factor 21 or an analog thereof is selected from polypeptides having an amino acid sequence at least 95% homologous to that of wild-type hFGF21, the flexible peptide linker is selected from L1, L2, L3, L4, and L5, the at least one rigid unit is selected from CTP1, CTP2, CTP3, CTP4, and CTP5, and the fusion partner is selected from vFcγ1, vFcγ2-1, vFcγ2-2, vFcγ2-3, and vFcγ4.

In some embodiments, the FGF21 fusion protein is selected from FP4A, FP4B, FP4C, FP4D, FP4E, FP4F, FP4G, FP4H, and FP4I.

In some embodiments, the FGF21 fusion protein comprises an amino acid sequence of SEQ ID NO: 8.

In some embodiments, the FGF21 fusion protein is a protein encoded by a nucleotide sequence of SEQ ID NO: 9.

In some embodiments, the therapeutically effective amount of the FGF21 fusion protein is from 5 mg to 400 mg. For example, the therapeutically effective amount of the FGF21 fusion protein is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, and 400 mg.

In some embodiments, the therapeutically effective amount of the FGF21 fusion protein is from 10 mg to 300 mg.

In some embodiments, the therapeutically effective amount of the FGF21 fusion protein is from 15 mg to 100 mg. For example, the therapeutically effective amount of the FGF21 fusion protein is 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg.

In some embodiments, the therapeutically effective amount of the FGF21 fusion protein is from 25 mg to 200 mg. For example, the therapeutically effective amount of the FGF21 fusion protein is 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, and 200 mg.

In some embodiments, the therapeutically effective amount of the FGF21 fusion protein is from 50 mg to 300 mg. For example, the therapeutically effective amount of the FGF21 fusion protein is 50 mg, 55 mg, 60 mg, 65 mg, 70 mg, 75 mg, 80 mg, 85 mg, 90 mg, 95 mg, 100 mg, 105 mg, 110 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

In some embodiments, the therapeutically effective amount of the FGF21 fusion protein is from 75 mg to 150 mg. For example, the therapeutically effective amount of the FGF21 fusion protein is 75 mg, 80 mg, 85 mg, 90 mg, 95 mg, 100 mg, 105 mg, 110 mg, 120 mg, 125 mg, 130 mg, 135 mg, 140 mg, 145 mg, and 150 mg.

In some embodiments, the therapeutically effective amount of the FGF21 fusion protein is from 15 mg to 60 mg. For example, the therapeutically effective amount of the FGF21 fusion protein is 15 mg, 20 mg, 25 mg, 30 mg, 35 mg, 40 mg, 45 mg, 50 mg, 55 mg, and 60 mg.

In some embodiments, the therapeutically effective amount of the FGF21 fusion protein is 10 mg or more (e.g., from 10 mg to 200 mg).

In some embodiments, the therapeutically effective amount of the FGF21 fusion protein is 15 mg or more (e.g., from 15 mg to 200 mg).

In some embodiments, the therapeutically effective amount of the FGF21 fusion protein is 20 mg or more (e.g., from 20 mg to 200 mg).

In some embodiments, the therapeutically effective amount of the FGF21 fusion protein is 25 mg or more (e.g., from 25 mg to 200 mg).

In some embodiments, the therapeutically effective amount of the FGF21 fusion protein is 30 mg or more (e.g., from 30 mg to 200 mg).

In some embodiments, the therapeutically effective amount of the FGF21 fusion protein is 35 mg or more (e.g., from 35 mg to 200 mg).

In some embodiments, the therapeutically effective amount of the FGF21 fusion protein is 37.5 mg or more (e.g., from 37.5 mg to 200 mg).

In some embodiments, the therapeutically effective amount of the FGF21 fusion protein is from 15 mg to 37.5 mg. For example, the therapeutically effective amount of the FGF21 fusion protein is selected from 15 mg, 17.5 mg, 20 mg, 22.5 mg, 25 mg, 27.5 mg, 30 mg, 32.5 mg, 35 mg, and 37.5 mg.

In some embodiments, the therapeutically effective amount of the FGF21 fusion protein is administered at least once every four weeks, e.g., once every four weeks, once every three weeks, once every two weeks, twice every three weeks, once a week, three times every two weeks, twice a week, or three times a week.

In some embodiments, the therapeutically effective amount of the FGF21 fusion protein is administered at least once every week, e.g., once a week, twice a week, or three times a week.

In some embodiments, the therapeutically effective amount of the FGF21 fusion protein is administered at least twice a week.

In some embodiments, the therapeutic effective amount of FGF21 fusion protein is administered three times a week, four times a week, five times a week, six times a week, or daily.

In some embodiments, the therapeutically effective amount of the FGF21 fusion protein is selected from 25 mg, 50 mg, 100 mg, 150 mg, and 200 mg.

In some embodiments, the therapeutically effective amount of the FGF21 fusion protein is selected from 50 mg, 75 mg, 100 mg, 150 mg, 200 mg, and 300 mg.

In some embodiments, the therapeutically effective amount of the FGF21 fusion protein is selected from 75 mg, 100 mg, and 150 mg, wherein the therapeutically effective amount of the FGF21 fusion protein is administered twice a week. For example, 75 mg, 100 mg, or 150 mg of the FGF21 fusion protein is administered to a subject in need thereof on Day 1 (D1), D4, D8, D11, D15, D18, D22, and D25, etc., for a total of 8 times or more by, e.g., subcutaneous injection.

In some embodiments, 15 mg of the FGF21 fusion protein is administered once a week. In some embodiments, 25 mg of the FGF21 fusion protein is administered once a week. In some embodiments, 25 mg of the FGF21 fusion protein is administered once every two weeks. In some embodiments, 37.5 mg of the FGF21 fusion protein is administered once every two weeks.

In some embodiments, the therapeutically effective amount of the FGF21 fusion protein is selected from 50 mg, 100 mg, and 150 mg, and wherein the therapeutically effective amount of the FGF21 fusion protein is administered twice a week.

In some embodiments, the FGF21 fusion protein is administrated by parenteral injection.

In some embodiments, the FGF21 fusion protein is administrated by subcutaneous or intravenous injection.

In some embodiments, the FGF21 fusion protein is administrated by subcutaneous injection and the injection site is at the abdominal wall, e.g., with <1.5 mL per injection point.

In some embodiments, the therapeutically effective amount of the FGF21 fusion protein is selected from 25 mg, 50 mg, 75 mg, 100 mg, 125 mg, 150 mg, and 200 mg.

In some embodiments, the therapeutically effective amount of the FGF21 fusion protein is selected from 50 mg, 75 mg, 100 mg, 150 mg, 200 mg, 250 mg, and 300 mg.

In some embodiments, the therapeutically effective amount of the FGF21 fusion protein is selected from 75 mg, 100 mg, and 150 mg, wherein the therapeutically effective amount of the FGF21 fusion protein is administered twice a week.

In some embodiments, the therapeutically effective amount of the FGF21 fusion protein is selected from 15 mg, 25 mg, and 37.5 mg, wherein the therapeutically effective amount of the FGF21 fusion protein is administered twice a week.

In some embodiments, the therapeutically effective amount of the FGF21 fusion protein is selected from 35 mg, 37.5 mg, 40 mg, 42.5 mg, 45 mg, and 50 mg, wherein the therapeutically effective amount of the FGF21 fusion protein is administered once every two weeks.

In some embodiments, the therapeutically effective amount of the FGF21 fusion protein is 15 mg administered once a week; the therapeutically effective amount of the FGF21 fusion protein is 25 mg administered once a week; the therapeutically effective amount of the FGF21 fusion protein is 25 mg administered once every two weeks; or the therapeutically effective amount of the FGF21 fusion protein is 37.5 mg administered once every two weeks.

In some embodiments, the therapeutically effective amount of the FGF21 fusion protein is selected from 50 mg, 100 mg, and 150 mg, wherein the therapeutically effective amount of the FGF21 fusion protein is administered twice a week.

In some embodiments, a pharmaceutical composition comprising the FGF21 fusion protein and at least one pharmaceutically acceptable carrier is administered.

In some embodiments, the concentration of the FGF21 fusion protein in the pharmaceutical composition is from 5 to 30 mg/ml, e.g., about 25 mg/ml.

In some embodiments, the disease or disorder is NAFLD. In some embodiments, the disease or disorder is NASH. In some embodiments, the disease or disorder is dyslipidemia.

An hFGF21 fusion protein may be administered, for example, in various manners, such as orally, topically, rectally, parenterally, by inhalation spray, or via an implanted reservoir, although the most suitable route in any given case will depend on the particular host, and nature and severity of the conditions for which the active ingredient is being administered.

In some embodiments, a pharmaceutically acceptable composition comprising an hFGF21 fusion, e.g., a reconstituted aqueous solution of hFGF21 fusion, can be drawn with a syringe according to the dosage for injection, such as for subcutaneous injection. The injection site on a subject may be at the abdominal wall, with, e.g., <=1.5 mL per injection point.

In some embodiments, the therapeutically effective amount of the FGF21 fusion protein is administered for a total of one to ten times, i.e., a total of one to ten doses of the therapeutically effective amount of the FGF21 fusion protein is administered to the subject over a period of time, e.g., a day, one week, two weeks, three weeks, four weeks, etc. In some embodiments, the therapeutically effective amount of the FGF21 fusion protein is administered for a total of at least one time. In some embodiments, the therapeutically effective amount of the FGF21 fusion protein is administered for a total of at least one time, two times, three times, four times, five times, six times, seven times, eight times, nine times, and ten times. In some embodiments, the therapeutically effective amount of the FGF21 fusion protein is administered for a total of at least 5 times. In some embodiments, the therapeutically effective amount of the FGF21 fusion protein is administered for a total of at least 8 times. In some embodiments, the therapeutically effective amount of the FGF21 fusion protein is administered for more than ten times, as the condition of the subject may require.

hFGF21 Fusion Proteins

In some embodiments, the hFGF21 fusion protein as disclosed herein is a highly glycosylated human fibroblast growth factor 21 (hFGF21) fusion protein, comprising, in the order from the N-terminus to C-terminus, wild-type human fibroblast growth factor 21 or an analog thereof, a flexible peptide linker (denoted as L), at least one rigid unit comprising the carboxyl terminal peptide of human chorionic gonadotropin β3 subunit (hereinafter referred to as (CTP) n, wherein n is 1, 2, 3, 4, or 5, etc.) and a fusion ligand (such as an immunoglobulin, an Fc fragment thereof, an albumin, or a transferrin).

In some embodiments, the hFGF21 fusion protein disclosed herein comprises a wild-type hFGF21 polypeptide, wherein the wild-type hFGF21 polypeptide comprises an amino sequence as shown in SEQ ID NO: 1 from which amino acids 1-28 (a leader peptide) are removed; or an isoform of the sequence as shown in SEQ ID NO: 1 from which amino acids 1-28 (a leader peptide) are removed and having a G141S or L174P substitution.

In some embodiments, the hFGF21 fusion protein disclosed herein comprises an hFGF21 analog, for example, an hFGF21 analog having one or more amino acid deletions, insertions, additions, or substitutions relative to its wild-type sequence, or a truncated form having one or more amino acid deletions at the N- or C-terminus. Preferably, the amino acid sequence of an hFGF21 analog is at least 70% homologous to that of wild-type hFGF21; more preferably, the amino acid sequence of an hFGF21 analog is at least 80% homologous to that of wild-type hFGF21; more preferably, the amino acid sequence of an hFGF21 analog is at least 90% (e.g., 90%, 91%, 92%, 93%, 94%, 95%, 96%, 97%, 98%, or 99%) homologous to that of wild-type hFGF21; and most preferably, the amino acid sequence of an hFGF21 analog is at least 95% (e.g., 95%, 96%, 97%, 98%, or 99%) homologous to that of wild-type hFGF21.

The flexible peptide linker is preferably non-immunogenic and can generate sufficient distance between hFGF21 and the fusion ligand to minimize the steric effects between them. In some embodiments, a flexible peptide linker consists of two or more amino acid residues selected from Gly (G), Ser(S), Ala (A), and Thr (T).

Preferably, the flexible peptide linker comprises amino acid residues G and S. The flexible peptide linker preferably has the structural formula of (GS) a (GGS) b (GGGS) c (GGGGS) d (SEQ ID NO: 10), wherein each of a, b, c, and d is an integer equal to or greater than 0, and a+b+c+d_1.

In some embodiments, the peptide linker is selected from L1, L2, L3, L4, L5, and L6 as shown below:

L1:
(SEQ ID NO: 11)
GGGGS;
L2:
(SEQ ID NO: 12)
GSGGGSGGGGSGGGGS;
L3:
(SEQ ID NO: 13)
GSGGGGSGGGGSGGGGSGGGGSGGGGS;
L4:
(SEQ ID NO: 14)
GGGGSGGGGSGGGGSGGGGSGGGGSGGGGSGGGGS;
L5:
(SEQ ID NO: 15)
GGGSGGGSGGGSGGGSGGGS;
and
L6:
(SEQ ID NO: 16)
GGSGGSGGSGGS

In some embodiments, a rigid unit is selected from a sequence consisting of carboxyl-terminal amino acids 113 to 145 of human chorionic gonadotropin β3 subunit, or a truncated sequence thereof. For example, a CTP rigid unit comprises the amino acid sequence as shown in SEQ ID NO: 2 or a truncated sequence thereof.

Preferably, a CTP rigid unit comprises at least 2 glycosylation sites. For example, in a preferred embodiment, the CTP rigid unit contains 2 glycosylation sites. Illustratively, the CTP rigid unit contains N-terminal 10 amino acids of SEQ ID NO: 2, i.e., SSSS*KAPPPS*(SEQ ID NO: 17); alternatively, a CTP rigid unit contains C-terminal 14 amino acids of SEQ ID NO: 2, i.e., S*RLPGPS*DTPILPQ (SEQ ID NO: 18). As another example, a CTP rigid unit contains 3 glycosylation sites. Illustratively, a CTP rigid unit contains N-terminal 16 amino acids of SEQ ID NO: 2, i.e., SSSS*KAPPPS*LPSPS*R (SEQ ID NO: 19). As another example, a CTP rigid unit contains 4 glycosylation sites. Illustratively, the CTP rigid unit contains 28, 29, 30, 31, 32, or 33 amino acids, starting from position 113, 114, 115, 116, 117, or 118 and ending at position 145 of the human chorionic gonadotropin beta subunit. For instance, a CTP rigid unit contains 28 amino acids: SSSS*KAPPPS*LPSPS*RLPGPS*DTPILPQ (SEQ ID NO: 2). The symbol * denotes a glycosylation site. Each possibility represents a separate embodiment of the present disclosure.

In some embodiments, the amino acid sequence of the CTP rigid unit provided by the present disclosure is at least 70% homologous to that of native CTP. In other embodiments, the amino acid sequence of the CTP rigid unit provided by the present disclosure is at least 80% homologous to that of native CTP. In other embodiments, the amino acid sequence of the CTP rigid unit provided by the present disclosure is at least 90% (e.g., 90%, 91%, 92%, 93%, 94%, 95%, 96%, 97%, 98%, or 99%) homologous to that of native CTP. In other embodiments, the amino acid sequence of the CTP rigid unit provided by the present disclosure is at least 95% (e.g., 95%, 96%, 97%, 98%, or 99%) homologous to that of native CTP.

Illustratively, the CTP rigid unit of the present disclosure may preferably comprise one or more of the following sequences:

CTP1:
(SEQ ID NO: 2)
SSSSKAPPPSLPSPSRLPGPSDTPILPQ;
CTP2:
(SEQ ID NO: 20)
PRFQDSSSSKAPPPSLPSPSRLPGPSDTPILPQ;
CTP3:
(SEQ ID NO: 17)
SSSSKAPPPS;
CTP4:
(SEQ ID NO: 18)
SRLPGPSDTPILPQ;
CTP5:
(SEQ ID NO: 19)
SSSSKAPPPSLPSPSR

In some embodiments, the hFGF21 fusion protein comprises one CTP rigid unit as described above. The fusion protein of the present disclosure may comprise more than one, preferably 2, 3, 4, or 5, CTP rigid units as described above.

In some embodiments, the hFGF21 fusion protein comprises three CTP3 rigid units: SSSSKAPPPSSSSSKAPPPSSSSSKAPPPS (SEQ ID NO: 21) (CTP3-CTP3-CTP3, or expressed as (CTP3) 3). In some embodiments, the fusion protein comprises two CTP5 rigid units: SSSSKAPPPSLPSPSRSSSSKAPPPSLPSPSR (SEQ ID NO: 22) (CTP5-CTP5, or expressed as (CTP5) 2).

In some embodiments, the fusion ligand is an immunoglobulin, an Fc fragment thereof, an albumin, or a transferrin. The fusion ligand is preferably an immunoglobulin Fc fragment. The Fc fragment is preferably selected from the group consisting of Fc fragments of human immunoglobulin IgG, IgM, IgA, and variants thereof, more preferably from the group consisting of Fc fragments of human IgG1, IgG2, IgG3, and IgG4 and variants thereof.

In some embodiments, a human IgG Fc variant (denoted as vFc) comprises at least one amino acid modification in the wild-type human IgG Fc. Preferably, the Fc variant is non-lytic and exhibits minimal Fc-mediated adverse side effects (e.g., ADCC and CDC effects) and/or enhanced binding affinity to the FcRn receptor.

In some embodiments, the human IgG Fc variant is selected from:
(i) vFcγ1: hinge, CH2 and CH3 regions of human IgG1 with mutations Leu234Val, Leu235Ala, and Pro331Ser (the amino acid sequence as shown in SEQ ID NO: 3);
(ii) vFcγ2-1: hinge, CH2 and CH3 regions of human IgG2 with mutation Pro331Ser (the amino acid sequence as shown in SEQ ID NO: 4);
(iii) vFcγ2-2: hinge, CH2 and CH3 regions of human IgG2 with mutations Thr250Gln and Met428Leu (the amino acid sequence as shown in SEQ ID NO: 5);
(iv) vFcγ2-3: hinge, CH2 and CH3 regions of human IgG2 with mutations Pro331Ser, Thr250Gln, and Met428Leu (the amino acid sequence as shown in SEQ ID NO: 6); and
(iv) vFcγ4: hinge, CH2 and CH3 regions of human IgG4 with mutations Ser228Pro and Leu235Ala (the amino acid sequence as shown in SEQ ID NO: 7).

In a preferred embodiment of the present disclosure, the hFGF21 fusion protein comprises the amino acid sequence as shown in SEQ ID NO: 8 (FP4I):

```
His Pro Ile Pro Asp Ser Ser Pro Leu Leu Gln Phe Gly Gly Gln Val
1 5 10 15
Arg Gln Arg Tyr Leu Tyr Thr Asp Asp Ala Gln Gln Thr Glu Ala His
              20 25 30
Leu Glu Ile Arg Glu Asp Gly Thr Val Gly Gly Ala Ala Asp Gln Ser
        35 40 45
Pro Glu Ser Leu Leu Gln Leu Lys Ala Leu Lys Pro Gly Val Ile Gln
    50 55 60
Ile Leu Gly Val Lys Thr Ser Arg Phe Leu Cys Gln Arg Pro Asp Gly
65 70 75 80
Ala Leu Tyr Gly Ser Leu His Phe Asp Pro Glu Ala Cys Ser Phe Arg
                85 90 95
Glu Leu Leu Leu Glu Asp Gly Tyr Asn Val Tyr Gln Ser Glu Ala His
          100 105 110
Gly Leu Pro Leu His Leu Pro Gly Asn Lys Ser Pro His Arg Asp Pro
        115 120 125
Ala Pro Arg Gly Pro Ala Arg Phe Leu Pro Leu Pro Gly Leu Pro Pro
    130 135 140
Ala Pro Pro Glu Pro Pro Gly Ile Leu Ala Pro Gln Pro Pro Asp Val
145 150 155 160
Gly Ser Ser Asp Pro Leu Ser Met Val Gly Pro Ser Gln Gly Arg Ser
                  165 170 175
Pro Ser Tyr Ala Ser Gly Ser Gly Gly Gly Gly Ser Gly Gly Gly Gly
                180 185 190
Ser Gly Gly Gly Gly Ser Gly Gly Gly Gly Ser Gly Gly Gly Gly Ser
            195 200 205
Ser Ser Ser Ser Lys Ala Pro Pro Pro Ser Leu Pro Ser Pro Ser Arg
        210 215 220
Leu Pro Gly Pro Ser Asp Thr Pro Ile Leu Pro Gln Val Glu Cys Pro
225 230 235 240
Pro Cys Pro Ala Pro Pro Val Ala Gly Pro Ser Val Phe Leu Phe Pro
                    245 250 255
Pro Lys Pro Lys Asp Gln Leu Met Ile Ser Arg Thr Pro Glu Val Thr
            260 265 270
Cys Val Val Val Asp Val Ser His Glu Asp Pro Glu Val Gln Phe Asn
      275 280 285
Trp Tyr Val Asp Gly Val Glu Val His Asn Ala Lys Thr Lys Pro Arg
    290 295 300
Glu Glu Gln Phe Asn Ser Thr Phe Arg Val Val Ser Val Leu Thr Val
305 310 315 320
Val His Gln Asp Trp Leu Asn Gly Lys Glu Tyr Lys Cys Lys Val Ser
                      325 330 335
Asn Lys Gly Leu Pro Ala Ser Ile Glu Lys Thr Ile Ser Lys Thr Lys
              340 345 350
Gly Gln Pro Arg Glu Pro Gln Val Tyr Thr Leu Pro Pro Ser Arg Glu
          355 360 365
Glu Met Thr Lys Asn Gln Val Ser Leu Thr Cys Leu Val Lys Gly Phe
      370 375 380
Tyr Pro Ser Asp Ile Ala Val Glu Trp Glu Ser Asn Gly Gln Pro Glu
385 390 395 400
Asn Asn Tyr Lys Thr Thr Pro Pro Met Leu Asp Ser Asp Gly Ser Phe
                    405 410 415
Phe Leu Tyr Ser Lys Leu Thr Val Asp Lys Ser Arg Trp Gln Gln Gly
              420 425 430
Asn Val Phe Ser Cys Ser Val Leu His Glu Ala Leu His Asn His Tyr
        435 440 445
Thr Gln Lys Ser Leu Ser Leu Ser Pro Gly Lys
    450 455
```

In some embodiments, the hFGF21 fusion protein of the present disclosure is glycosylated. Preferably, the hFGF21 fusion protein is glycosylated by expressing the same in mammalian cells. More preferably, the hFGF21 fusion protein is glycosylated by expressing the same in Chinese hamster ovary cells.

In a preferred embodiment of the present disclosure, the hFGF21 fusion protein is encoded by the nucleic acid sequence as shown in SEQ ID NO: 9:

caccctattcccgatagctcccccctcctgcagttcggaggccaggtgaggcagcggtac 60
ctgtacaccgacgacgctcagcagaccgaagctcacctggagatcagggaggatggaacc 120
gtcggcggagctgctgaccagtcccccgagagcctgctgcagctgaaggccctgaagccc 180
ggagtcatccagatcctgggcgtgaagacctcccggtttctgtgtcagcggcccgatgga 240
gccctgtacggctccctgcattttgaccccgaggcctgtagcttcagggagctgctgctg 300
gaagacggctacaacgtgtaccagagcgaagctcacggactgcccctgcacctgcctggc 360
aacaaatcccctcacagggaccccgctcccaggggacctgccaggttectgcctctgccc 420
ggactgcctcctgctcctcccgaacctcctggcatcctegctcctcagccccctgatgtc 480
ggcagcagegaccctctgtccatggtcggccccagccaaggcaggagcccttcctacgct 540
tccggatccggtggcggtggctccggtggaggcggaagcggcggtggaggatcaggcggt 600
ggaggtagcggcggaggcggtagetccagctctagtaaagctccccctccttccctgccc 660
tcaccctcaagactgcctggaccttccgacactcccatcctgccacaggtggagtgccct 720
ccatgtccagcaccccctgtcgcaggtccatctgtgttcctgtttccacccaagcctaaa 780
gaccagctgatgatctcccgcaccccagaagtcacctgtgtggtcgtggatgtgagccat 840
gaagaccccgaggtccagttcaattggtacgtggatggcgtcgaggtgcacaacgctaag 900
acaaaacctagagaagagcagttcaactctacctttcgcgtcgtgagtgtgctgacagtc 960
gtgcaccaggactggctgaatggcaaggagtataagtgcaaagtgagcaacaaaggactg 1020
cctgcctcaatcgaaaagactatttccaagaccaaaggacagccaagagagccccaggtg 1080
tacaccctgcctccaagccgcgaagagatgactaaaaatcaggtctctctgacctgtctg 1140
gtgaaggggttttatcctagtgatatcgccgtggaatgggagtcaaacggtcagccagag 1200
aacaattacaagaccacaccccctatgctggacagcgatgggtctttctttctgtatagc 1260
aaactgacagtggacaagtctcggtggcagcagggtaacgtcttctcttgcagtgtgctg 1320
cacgaagcactgcacaatcattacacccagaagtcactgtcactgagcccaggaaaatga 1380

The present disclosure provides a series of hFGF21 fusion proteins as shown in Table 1. Each of FP4A, FP4B, FP4C, FP4D, FP4E, FP4F, FP4G, FP4H, and FP4I comprises wild-type hFGF21 or its analog, a flexible peptide linker (L), a CTP rigid unit, and an Fc variant (vFc), while FP4J does not comprise a CTP rigid unit.

TABLE 1
Exemplary hFGF21 fusion proteins

| | Components of hFGF21 fusion proteins |
|---|---|
| Code | (from N-terminus to C-terminus) |
| FP4A | ^{P199G}hFGF21-L1-CTP¹-vFcγ₂₋₃ |
| FP4B | hFGF21(HPIP⁻)-L2-CTP¹-vFcγ₂₋₃ |
| FP4C | ^{K150R}hFGF21-L3-CTP⁵-vFcγ₁ |
| FP4D | ^{G202A}hFGF21-L4-CTP⁴-vFcγ₄ |
| FP4E | ^{A109T}hFGF21-L1-CTP³-CTP³-CTP³-vFcγ₂₋₂ |
| FP4F | hFGF21-L5-CTP⁵-CTP⁵-vFcγ₂₋₃ |
| FP4G | ^{Q55C}hFGF21-L4-CTP³-vFcγ₂₋₂ |
| FP4H | ^{L126R}hFGF21-L6-CTP²-vFcγ₄ |
| FP4I | ^{L174P}hFGF21-L3-CTP¹-vFcγ₂₋₃ |
| FP4J | ^{L174P}hFGF21-L3-vFcγ₂₋₃ |

Preparation of hFGF21 Fusion Proteins

hFGF21 Fusion proteins of the present disclosure can be prepared as described in WO 2018/032785 A1 (e.g., Examples 1-3 therein). For example, an hFGF21 fusion protein of the present disclosure is typically prepared by biosynthetic methods. Based on the amino acid and nucleotide sequences of the present disclosure, one skilled in the art can prepare suitable nucleic acids by various known methods, for example, but not limited to, PCR, DNA synthesis, etc. For specific methods, see J. Sambrook, Molecular Cloning: A Laboratory Manual. As an embodiment of the present disclosure, the nucleic acid sequence of the present disclosure can be constructed by segmentally synthesizing nucleotide sequences and then performing overlap extension PCR.

The present disclosure also provides an expression vector comprising a sequence encoding an hFGF21 fusion protein of the present disclosure and an expression regulatory sequence operably linked thereto. The term “operably linked” or “operably linked to” means a condition in which some portions of a linear DNA sequence are capable of regulating or controlling the activity of other portions of the same linear DNA sequence. For instance, a promoter is operably linked to a coding sequence if the promoter controls the transcription of the sequence.

The expression vector may be a commercially available vector such as, but not limited to, pcDNA3, pIRES, pDR, pUC18, or the like, which can be used in a eukaryotic cell expression system. One skilled in the art can select a suitable expression vector based on the host cell.

The coding sequence of the fusion protein of the present disclosure may be introduced into suitable restriction sites by one skilled in the art by restriction enzyme cleavage and splicing according to a conventional method based on the restriction enzyme map of a known expression vector, to produce the recombinant expression vector of the present disclosure.

The disclosure also provides a host cell expressing an hFGF21 fusion protein of the disclosure comprising a coding sequence of a fusion protein of the present disclosure. The host cell is preferably a eukaryotic cell such as, but not limited to, CHO cells, COS cells, 293 cells, RSF cells, and the like. In a preferred embodiment of the present disclosure, the cell is a CHO cell, which can well express an hFGF21 fusion protein of the present disclosure to obtain a fusion protein having good binding activity and good stability.

The present disclosure also provides a method for producing a fusion protein of the present disclosure by using recombinant DNA, including the steps of:
1) providing a nucleic acid sequence encoding a fusion protein;
2) inserting the nucleic acid sequence of 1) into a suitable expression vector to obtain a recombinant expression vector;
3) introducing the recombinant expression vector of 2) into a suitable host cell;
4) growing the transformed host cell under conditions suitable for expression; and
5) collecting the supernatant and purifying the fusion protein product.

The coding sequence can be introduced into a host cell by various techniques known in the art such as, but not limited to, calcium phosphate precipitation, protoplast fusion, lipofection, electroporation, microinjection, reverse transcription, phage transduction, and method using alkali metal ions.

For the culture and expression of host cells, see Olander RM Dev Biol Stand, 1996, 86:338. The cells and debris in the suspension can be removed by centrifugation and the supernatant is collected. Identification can be performed by agarose gel electrophoresis.

A fusion protein obtained as described above can be purified to a substantially uniform nature, for example, showing a single band on SDS-PAGE electrophoresis. For example, when the recombinant protein is expressed by secretion, a commercially available ultrafiltration membrane such as products from Millipore, Pellicon, etc. can be used to separate the protein. The supernatant is firstly to be concentrated. The concentrated supernatant may be further purified by gel chromatography or by ion exchange chromatography, such as anion exchange chromatography (DEAE, etc.) or cation exchange chromatography. The gel matrix may be a matrix commonly used for protein purification such as agarose, dextran, polyamide, and the like. The Q- or SP-group is a preferred ion exchange group. The purified product may be further finely purified by methods such as hydroxyapatite adsorption chromatography, metal chelate chromatography, hydrophobic interaction chromatography and reversed-phase high performance liquid chromatography (RP-HPLC), and the like. All of the above purification steps can be used in different combinations to ultimately obtain proteins with a substantially uniform purity.

The expressed fusion protein can be purified by using an affinity chromatography column containing an antibody, receptor, or ligand specific for the fusion protein. Depending on the nature of the affinity column used, the fusion polypeptide bound to the affinity column can be eluted by using conventional methods such as high salt buffer, pH change, and the like. Alternatively, the fusion protein may also contain one or more polypeptide fragments as a protein tag at the amino terminus or carboxyl terminus. Any suitable label can be used in the present disclosure. For example, the label may be FLAG, HA, HA1, c-Myc, 6-His or 8-His, and the like. These tags can be used to purify the fusion protein.

### Pharmaceutical Compositions

Some embodiments of the present disclosure relate to a pharmaceutical composition comprising a recombinant human FGF21-FC fusion protein and a pharmaceutically acceptable carrier, excipient, or diluent.

In some embodiments, the pharmaceutical composition comprises 10 to 30 mg of an FGF21-FC fusion protein. For example, a pharmaceutical composition of the present disclosure comprises more than 25 mg/vial of a human FGF21-FC fusion protein.

In some embodiments, a vial is used to contain a lyophilized human FGF21-Fc fusion protein. The FGF21-Fc fusion protein can be reconstituted with water for injection. For example, 25 mg of FGF21-Fc fusion protein in a vial can be reconstituted with 1 ml of water for injection to obtain a ~25 mg/mL/vial reconstituted solution.

### EXAMPLES

The following examples are provided to describe the disclosure in greater detail. They are intended to illustrate, not to limit, the disclosure.

### Example 1: Single-Dose Pharmacokinetic Study of FP4I in SD Rats

A single subcutaneous injection of a recombinant human FGF21-FC fusion protein (FP4I) was administered to rats at a dose of 5 mg/kg to study the pharmacokinetic profiles of the drug concentration in serum over time.

Specifically, a total of 6 SD rats, half male and half female, were subcutaneously administered with 5 mg/kg of FP4I at a volume of 1.25 mL/kg. Blood was collected from the rats at 0 h before the administration of FP4I, and at 2 h, 6 h, 12 h, 24 h, 36 h, 48 h, 72 h, 96 h, 168 h, and 240 h after the start of the administration of FP4I. An ELISA method was used to detect and analyze the concentration of FGF21-Fc in serum, and a non-compartmental model analytical method was used to analyze relevant pharmacokinetic parameters using the WinNonlin Phoenix (v6.4, Pharsight Company) software.

The results of certain pharmacokinetic parameters are shown in Table 2. The results showed that the in vivo terminal elimination half-life T_{1/2} of the FGF21-Fc in the 5 mg/kg dose group was 30.2 hours, and the mean residence time (MRT) was 56.1 hours.

TABLE 2
Pharmacokinetic parameters of FGF21-Fc after
subcutaneous injection of 5 mg/kg of FP4I in SD rats
PK Parameters

| Tₘₐₓ | Cₘₐₓ | AUC_{INF}_{_{—}}_{obs} | AUCₗₐₛₜ | T_{1/2}_{_{—}}_{z} | Cl_{_{—}}_{F}_{_{—}}_{obs} | V_{z}_{_{—}}_{F}_{_{—}}_{obs} | MRT_{INF}_{_{—}}_{obs} | MRTₗₐₛₜ |
|---|---|---|---|---|---|---|---|---|
| (h) | (μg/mL) | (h*μg/mL) | (h*μg/mL) | (h) | (mL/kg/h) | (mL/kg) | (h) | (h) |
| 24.0 | 16.3 | 1060 | 1050 | 30.2 | 4.81 | 209 | 56.1 | 54.4 |

### Example 2: Pharmacokinetic Study of FP4I in Cynomolgus Monkeys

A single subcutaneous injection of recombinant human FGF21-FC fusion protein (FP4I) was administered to cynomolgus monkeys at a dose of 5 mg/kg to study the pharmacokinetic profiles of the drug concentration in serum over time.

A total of 6 cynomolgus monkeys, half of which male and half female, were administered with 5 mg/kg of FP4I by subcutaneous injection at a volume of 1.25 mL/kg. Blood was collected from the cynomolgus monkeys at 0 h before the administration of FP4I and at 2 h, 6 h, 12 h, 24 h, 36 h, 48 h, 72 h, 96 h, 168 h, and 240 h after the start of the administration of FP4I. An ELISA method was used to detect and analyze the concentration of FGF21-Fc in serum, and a non-compartmental model analysis method was used to analyze relevant pharmacokinetic parameters using the WinNonlin Phoenix (v6.4, Pharsight Company) software.

The results of certain pharmacokinetic parameters are shown in Table 3. The results showed that the terminal elimination half-life T_{1/2} of the FGF21-Fc in the 5 mg/kg dose group was 39.2 hours, and the mean residence time (MRT) was 48.8 hours.

TABLE 3
Pharmacokinetic parameters of FGF21-Fc after
subcutaneous injection of FP4I in cynomolgus monkeys
PK Parameters

| Tₘₐₓ | Cₘₐₓ | AUC_{INF}_{_{—}}_{obs} | AUCₗₐₛₜ | T_{1/2}_{_{—}}_{z} | Cl_{_{—}}_{F}_{_{—}}_{obs} | V_{z}_{_{—}}_{F}_{_{—}}_{obs} | MRT_{INF}_{_{—}}_{obs} | MRTₗₐₛₜ |
|---|---|---|---|---|---|---|---|---|
| (h) | (μg/mL) | (h*μg/mL) | (h*μg/mL) | (h) | (mL/kg/h) | (mL/kg) | (h) | (h) |
| 24.0 | 31.8 | 1820 | 1800 | 39.2 | 2.88 | 162 | 48.8 | 46.8 |

### Example 3: Preclinical Studies of FGF21-Fc Fusion Protein

### 3.1 Pharmacodynamics

### 3.1.1 In Vitro Pharmacodynamics

An FGF21 responsive reporter gene cell line has been established by transfecting the human β-klotho gene and reporter constructs containing 5×UAS-firefly luciferase and GAL4 DNA-binding domain fused to ELK1 into HEK293T cells to evaluate the in vitro activity of FP4I. The results showed that FP4I can increase luciferase activity in cells, indicating that FP4I exhibited human FGF21 activity in vitro.

Binding Affinity Study of Recombinant Human FGF21-Fc Fusion Protein with FcRn Protein of Different Species

This study indicated that the recombinant human FGF21-Fc fusion protein FP4I had certain affinity with human FcRn, cynomolgus/rhesus FcRn, and rat and mouse FcRn, suggesting that FP4I had a relatively long half-life in the serum of these species.

Binding Affinity Study of Recombinant Human FGF21-Fc Fusion Protein with Recombinant Human FcγRI, FcγRIIB, FcγRIIIA and C1q Protein

This study indicated that FP4I had an extremely low affinity with FcγRI, FcγRIIB, FcγRIIIA and Clq, suggesting that FP4I may be able to avoid Fc-mediated antibody-dependent cellular cytotoxicity (ADCC) and complement-dependent cytotoxicity (CDC) effects.

### 3.1.2 In Vivo Pharmacodynamics

### Evaluation of the Efficacy of FP4I in Reversing Hepatic Steatosis in High-Fat Diet Induced Male Obese Mice

This study indicated that FP4I significantly ameliorated hepatic steatosis in high-fat induced obese mice. Results showed that FP4I administered at 4 mg/kg and 8 mg/kg significantly reduced the total TG (triglycerides) content in the liver of the mice. Histopathological examination showed that FP4I at the 4 mg/kg and 8 mg/kg alleviated liver steatosis and reduced liver lipids. In addition, FP4I at 8 mg/kg significantly reduced the serum alanine aminotransferase (ALT) and aspartate aminotransferase (AST) in mice.

### Efficacy of FP4I in NASH Cynomolgus Monkeys

This study showed that FP4I had liver protective effect on NASH cynomolgus monkeys. Results showed that, compared with baseline values, FP4I at a high-dose (7.5 mg/kg) significantly reduced liver fat content in cynomolgus monkeys (relative reduction of 29.89%, P<0.05). The results of histopathological examination showed that FP4I treatment at a medium dose (2.5 mg/kg) and a high dose (7.5 mg/kg) improved liver steatosis in cynomolgus monkeys.

### 3.2 Non-Clinical Pharmacokinetics

### 3.2.1 Absorption

### Pharmacokinetic Study in SD Rats

SD rats were administrated with 1 mg/kg, 5 mg/kg, and 25 mg/kg FP4I, respectively, by a single subcutaneous injection. The results showed that the increase of AUC_{INF_obs}, AUCₗₐₛₜ and Cₘₐₓ of FP4I in rat serum in the dose range of 1 mg/kg to 25 mg/kg was proportional to the dose increase.

Compared with the single intravenous injection group (5 mg/kg), the bioavailability in the single subcutaneous injection group (5 mg/kg) was 38.1% (based on AUC_{INF_obs}).

SD rats were administered with 5 mg/kg FP4I for 6 times, once every 3 days. The ratio of AUC₀₋₇₂ₕ comparing the first and last doses was 1:0.805, and the ratio of the first and last Cₘₐₓ was 1:0.827. These results indicated that there was no accumulation of FP4I in SD rats after 6 repeated dosing at 5 mg/kg.

### Pharmacokinetic Study in Cynomolgus Monkeys

Cynomolgus monkeys were administered with 1 mg/kg, 5 mg/kg, and 25 mg/kg FP4I by subcutaneous injection, respectively, or administered with 5 mg/kg FP4I by intravenous injection. There was no significant difference in the main pharmacokinetic parameters (AUC, Cₘₐₓ, Cl, t_{1/2_z}) between male and female animals in each group. The results showed that the increase of AUC_{INF_obs}, AUCₗₐₛₜ and Cₘₐₓ of FP4I in Cynomolgus monkey serum in the dose range of 1 mg/kg to 25 mg/kg was proportional to the dose increase.

Compared with the single intravenous injection group (5 mg/kg), the bioavailability in the single subcutaneous injection group (5 mg/kg) was 62.1% (based on AUC_{INF_obs}).

Cynomolgus monkeys were administered with 5 mg/kg FP4I for 6 times, once every 3 days. The ratio of AUC₀₋₇₂ h for the first and last doses was 1:0.730, and the ratio of Cₘₐₓ was 1:0.729. These results indicated that there was no accumulation of FP4I in cynomolgus monkeys after 6 repeated dosing at 5 mg/kg.

### 3.3 Toxicology

### 3.3.1 Safety Pharmacology

### Effects on the Central Nervous System of SD Rats

FP4I was administered by a single subcutaneous injection to SD rats and the effects of the test drug on the central nervous system of SD rats were examined. The study showed that the functional observation battery (FOB), forelimb grip strength, and body temperature of the subjects in each dosing group (100 mg/kg, 200 mg/kg, and 400 mg/kg (2 times a week, 8 times in total) had no significant difference compared with the vehicle group.

### Effects on the Cardiovascular System and Respiratory Frequency of Cynomolgus Monkeys

The effects of FP4I on the cardiovascular system and respiratory system were investigated with repeated administration of FP4I in toxicity tests in cynomolgus monkeys. The test results showed that compared with the vehicle control group, the ECG parameters, blood pressure, and respiratory frequency of the male and female animals in the low, medium, and high dose groups (50 mg/kg, 100 mg/kg, and 200 mg/kg, twice a week, for a total of 8 times) of the test drug product had no significant difference associated with the test drug product.

### 3.3.2 Single-Dose Toxicity in Rats

In a single-dose toxicity study of FP4I administered subcutaneously in SD rats, randomization was performed according to gender and body weight of the rats. There were 10 rats in each group, half male and half male, divided into the vehicle control group and the test product groups. In the control group, the rats were administered with a subcutaneous injection of the vehicle of 20 mL/kg, twice a day. In the test groups, the rats were administered with a subcutaneous injection of 400 mg/kg (20 mL/kg), twice a day, for a total dose of 800 mg/kg/day. The observation period was 14 days. No animals were found dead or dying during the experiment.

No vehicle control or test article-related abnormality in clinical observation was observed in all animals. The average body weight of male animals in the test article group was significantly lower than that in the vehicle control group on Day 7 and Day 14. These changes were presumably due to the pharmacodynamics effects of the test article and of non-toxic nature. No vehicle control or test article-related macroscopic findings were observed in all treated animals.

Under this experimental condition, the highest single subcutaneous injection of FP4I for SD rats with no-observed-adverse-effect level (NOAEL) was 800 mg/kg/day.

### 3.3.3 Single-Dose Toxicity in Cynomolgus Monkeys

In a single-dose toxicity study, FP4I was administered by subcutaneous injection in cynomolgus monkeys, according to a complete randomization, each group with 2 cynomolgus monkeys, one male and one female. The animals were divided into a vehicle control group and a test product group. The animals in the vehicle control group was subcutaneously injected with FP4I at 10 ml/kg, twice a day. The animals in the test group were injected with a subcutaneous administration dose of 200 mg/kg (with volume of 10 mL/kg), twice a day, with a total dose of 400 mg/kg/day. The observation period was 14 days. During the study, no vehicle control or test article-related changes were noted in clinical observation, body weight, food consumption, body temperature, ECG, hematology, coagulation, and clinical chemistry of male and female animals in the vehicle control or test article group. No obvious macroscopic findings were noted in all animals at necropsy at the end of observation period.

Under the experimental conditions, the highest single subcutaneous injection of FP4I for cynomolgus monkeys with no-observed-adverse-effect level (NOAEL) was 400 mg/kg/day.

### 3.3.4 Repeated Dose Toxicity Test in Rats

In a repeated dose toxicity test, FP4I was administered by subcutaneous injection in SD rats, at 100 mg/kg, 200 mg/kg, and 400 mg/kg (2 times a week, 8 times in total) for 4 consecutive weeks, with a four-week recovery period. During the study, no obvious test article-related toxicity change was observed. The NOAEL for this test was 400 mg/kg.

### 3.3.5 Repeated Dose Toxicity Test in Cynomolgus Monkeys

Cynomolgus monkeys were administered with subcutaneous injection of FP4I at 50 mg/kg, 100 mg/kg, and 200 mg/kg, twice a week, for a total of 8 times, with a 4-week recovery period. Only decreased body weight (pharmacodynamic related), and non-adverse reactions such as decreased IgG at dose ≥50 mg/kg, and slight renal tubular dilatation in female animals at dose ≥100 mg/kg were noted, and no significant toxicological change related to the test article was observed. The NOAEL obtained in this test was 200 mg/kg.

### 3.3.6 Immunotoxicity Test

This study was accompanied with pharmacokinetics study of FP4I in SD rats by single and repeated subcutaneous administration. After a single subcutaneous injection at 1, 5, and 25 mg/kg and intravenous injection at 5 mg/kg, 0% (0/6), 0% (0/6), 100% (6/6), and 0% (0/6) of the individuals, respectively, were detected positive for the anti-drug antibody. After repeated subcutaneous injections at 5 mg/kg, 50% (3/6) of the individuals were detected positive for the anti-drug antibody.

This study was accompanied with pharmacokinetics study of FP4I in cynomolgus monkeys by single and repeated subcutaneous administration. After a single subcutaneous injection of FP4I at 1, 5, and, 25 mg/kg and intravenous injection at 5 mg/kg, 17% (1/6), 17% (1/6), 67% (4/6), and 50% (3/6) of the individuals, respectively, were detected positive for the anti-drug antibody. After repeated subcutaneous injections at 5 mg/kg, 33% (2/6) of the individuals were detected positive for the anti-drug antibody.

In a toxicity test of subcutaneous injection of FP4I (100 mg/kg, 200 mg/kg, and 400 mg/kg) in SD rats for 4 weeks followed with a 4-week recovery period, 6/10 test animals in the vehicle control group developed anti-drug antibodies, and 5/10 test animals were positive for neutralizing antibodies. The test animals in the 100 mg/kg, 200 mg/kg, and 400 mg/kg groups were all positive for anti-drug antibodies and neutralizing antibodies.

In a toxicity test of subcutaneous injection of FP4I in cynomolgus monkeys for 4 weeks followed with a 4-week of recovery period, no test animals in the vehicle control group developed anti-drug antibodies. 3/10 test animals in the 50 mg/kg group were positive for anti-drug antibodies. 3/10 test animals in the 100 mg/kg group were positive for anti-drug antibodies, and 1/10 test animal was positive for neutralizing antibodies. 4/10 test animals in the 200 mg/kg group were positive for anti-drug antibodies, and 2 test animals were positive for neutralizing antibodies.

### 3.3.7 Hemolysis Study

The objective of this study was to assess the hemolytic potential of FP4I in vitro using red blood cells from a New Zealand white rabbit. FP4I at the concentration of 20 mg/mL did not cause hemolysis or RBC aggregation.

### 3.3.8 Administration Site Irritation Study in Cynomolgus Monkeys

This study was accompanied with a 4-week repeated dose toxicity study of FP4I via subcutaneous injection in cynomolgus monkeys with a 4-week recovery period. No test article-related irritation reaction was observed at the administration site.

### Example 4: Escalating Dosing Regimen Design

Nonclinical toxicity studies indicate that FGF21-Fc fusion protein has a broad safety window. In repeated-dose toxicity studies, SD rats were subcutaneously injected with FP4I at 100 mg/kg, 200 mg/kg, and 400 mg/kg each time, cynomolgus monkeys were injected subcutaneously with FP4I at 50 mg/kg, 100 mg/kg, and 200 mg/kg each time, each dosing twice a week (biw) for 4 consecutive weeks with a 4-week recovery period. The results showed that, except for some body weight decrease, there were no significant changes in toxicity related to FP4I.

The NOAEL doses for SD rats and cynomolgus monkeys in these studies were 400 mg/kg and 200 mg/kg, respectively. These NOAEL doses can be converted to human equivalent dose (HED) as 65 mg/kg and 81 mg/kg, respectively. For example, for a human patient of 60 kg, the doses are 3900 mg and 4860 mg, respectively. Considering the characteristics of biological products and taking the safety factor of 100, the starting doses for human patients are about 40 mg and about 50 mg.

In a pharmacodynamic study of a cynomolgus monkey model of NASH, the cynomolgus monkeys were administered with subcutaneous injections of FP4I at 0.8 mg/kg, 2.5 mg/kg, and 7.5 mg/kg, once per week (Qw), for 4 weeks of continuous administration. The results showed that 2.5 mg/kg and 7.5 mg/kg both improved hepatic steatosis and 7.5 mg/kg significantly reduced the content of liver fat. These results indicate that the minimum effective dose of FP4I for cynomolgus monkey is 2.5 mg/kg, and the corresponding HED is 1.01 mg/kg. The estimated dose for a human patient of 60 kg is about 60 mg. The effective dose of FP4I for cynomolgus monkey is 7.5 mg/kg, and the corresponding HED is 3.03 mg/kg. The estimated dose for a human patient of 60 kg is about 180 mg.

Pharmacokinetic studies showed that the FP4I drug exposure in SD rats and cynomolgus monkeys with single administration doses of 1 mg/kg, 5 mg/kg, and 25 mg/kg is proportional to the dose, the time to peak (Tₘₐₓ) was 24~30 h, and the terminal elimination half-life (T_{1/2_z}), in SD rats, was 27.0±1.66 h, 30.2±2.15 h, and 25.4±2.26 h, respectively, and in cynomolgus monkey, was 44.0±8.59 h, 39.2±5.26 h and 25.5±2.48 h, respectively. After 6 repeated dosing at 5 mg/kg, there was no obvious drug accumulation tendency in SD rats or cynomolgus monkeys.

### Example 5: Randomized Double-Blind Phase I Clinical Study of Single and Multiple Dosing

A randomized, double-blind phase I clinical study of single/multiple doses of recombinant human FGF21-Fc fusion protein (FP4I) for assessment of safety, tolerability, pharmacokinetics, immunogenicity, and efficacy in adult subjects are being conducted.

### Single Ascending Dose (SAD)

A total of 5 dose groups were set up in the clinical phase I trial of SAD study in human as follows: Group 1 (25 mg, pilot cohort), Group 2 (50 mg), Group 3 (100 mg), Group 4 (150 mg), Group 5 (200 mg), and higher dose groups (if needed). Healthy subjects were enrolled in this trial phase and each received only one study dose or placebo.

In Group 1, two subjects were enrolled, one received FP4I and one received placebo. Starting from group 2, each experimental group consisted of 8 subjects, including both male and female. Among them, the first 2 subjects were randomized to receive FP4I or placebo. After being reviewed and approved by SRC (Safety Review Committee), the remaining 6 subjects were randomized to receive FP4I or placebo in a ratio of 5:1.

Each subject is required to follow a study procedure, and undergo safety assessment and blood sampling per study schedule. The first stage of the study is conducted in a research center. Subjects can leave the research center with the approval of the investigator after completing the safety assessment in the morning of D7. After leaving the research center, the subject will return to the research center per study schedule to undertake safety assessment and blood sampling.

After all subjects in each dose group complete the D7 safety assessment, the SRC members would discuss and make the decision for dose escalation. After the decision is made, a higher-dose group study or an additional dose group can be started per protocol.

During the study period, the subjects will be closely monitored for adverse events. If there are adverse events not yet recovered or newly emerged in a subject by the end of the study, the investigators will continue to follow up with the subject, and will arrange the subject to return to the research center for relevant examinations, if necessary, until the adverse event returned to a clinically acceptable level, stabilized, or the subject fails to appear for the follow-up.

### Multiple Ascending Dose (MAD)

If SAD Cohort 3 (100 mg) is completed and is safe and well tolerated, upon receiving approval from the SRC, the MAD study can be initiated. The MAD study includes 3 dose groups: Group 1 (75 mg), Group 2 (100 mg), and Group 3 (150 mg), with biw administration. The study subjects are administered with the study drug on the following days: D1, D4, D8, D11, D15, D18, D22, and D25, for a total of 8 times, by subcutaneous injection. The dose and frequency of administration in the MAD study can be adjusted according to the results of the SAD study and determined by the SRC. If necessary, corresponding protocol revisions can be made.

The MAD phase will enroll overweight or obese subjects with elevated fasting triglyceride (≥2.0 mmol/L≤6.0 mmol/L), 8 in each group, including both male and female. Among them, the first 2 cases in each group will randomly receive FP4I or placebo in a ratio of 1:1. After SRC review and approval, the remaining 6 subjects will be given FP4I or placebo in a ratio of 5:1. The study drug administrated and follow-up study visit procedures are the same for the later 6 subjects as the first 2 subjects in a given dosing group.

Each subject is required to follow a study procedure and is subject to safety assessment and blood sampling. The first stage of this study is also conducted in a research center. Subjects can leave the research center with the approval of the investigator after completing the safety assessment on D31. After leaving the research center, the subject will return to the research center according to the study procedure and undertake the safety assessment and blood sampling. The subject will complete the study after finishing all the required tests and blood sampling on D64.

Dose advance will be approved by SRC after all subjects in each dose group have completed the D15 visit, and after the SRC members have discussed and reached a dose escalation decision, the study of the next higher dose group or a new dose group trial can be started.

During the study period, the subjects should be closely monitored for adverse events. If there are adverse events not yet recovered or newly emerged in a subject at the end of the study, the investigators will continue to follow up with the subject, and if necessary, will arrange the subject to return to the research center for relevant examinations, until the adverse event returned to a clinically acceptable level, stabilized, or the subject fails to appear for the follow-up.

### Study Subject

According to NMPA (National Medica Products Administration of China) guidelines, at least 8 evaluable subjects are recruited per dose group. About 34 to 42 healthy subjects will be enrolled in the SAD stage, about 24 overweight or obese subjects will be enrolled in the MAD stage, and a total of about 58-66 subjects will be enrolled in the studies.

### Study Drug

A recombinant human FGF21-Fc fusion protein for injection (FP4I), 25 mg/vial, was used in the SAD and MAD studies. The study drug is refrigerated at 2~8° C., and stored away from light.

### Drug Administration

The study subjects have fasted for at least 10 hours before administration. For each vial containing the recombinant human FGF21-Fc fusion protein for injection, the FGF21-Fc fusion protein is reconstituted with water for injection (1.1 mL) to obtain a 25 mg/mL/vial reconstituted solution. The reconstituted drug solution is drawn with a syringe according to the dosage for subcutaneous injection. The injection site is at the abdominal wall, with <1.5 mL per injection point.

### Study Subject Inclusion Criteria

1. The study subject must be able to understand and voluntarily sign a written informed consent, including the study drug and all research samples collection.
2. Male or female subjects aged 18 to 65 years (including the cutoff values).
3. Weight and Body Mass Index (BMI)
SAD study: male weight ≥50.0 kg, female weight ≥45.0 kg,
BMI is weight (kg)/height² (m²)=19~26 kg/m² (including the cutoff values).
MAD study: 26~35 kg/m² (with the cutoff values).
4. The subject (including partner) reported no plans to become pregnant from two weeks before screening until 6 months after the last study dose, and voluntarily use non-pharmacological contraception.

### Study Subject Exclusion Criteria

1. Have received any vaccine within 28 days prior to the first dose of the study drug.
2. Reported a history of allergies in the past, especially allergic to protein drugs or food.
3. Smoked ≥5 cigarettes per day within 3 months before screening for the clinical study, or who could not stop using any tobacco products during the trial.
4. Reported history of alcohol abuse 3 months prior to the study screening (14 units of alcohol per week: 1 unit ≈360 mL of beer or 45 mL 40% alcohol or 150 mL of wine); or cannot stop taking any alcoholic beverage during the clinical trial, or those who tested positive for alcohol.
5. Excessive consumption of tea, coffee, or caffeinated beverages within 3 months prior to screening (more than 8 cups of the beverages per day, 1 cup as 250 mL), or ingested any caffeinated food or drink (such as coffee, strong tea, chocolate, cola, etc.) within 48 hours before the first dose of the study drug.
6. Reported history of drug abuse within 1 year prior to screening or a positive drug screening test during the trial period.
7. A definitive diagnosis of type 1 diabetes or type 2 diabetes.
8. Abnormal examination results during the screening period are clinically significant and meet the following criteria:
   Alanine aminotransferase (ALT), aspartate aminotransferase (AST) or total bilirubin (TBIL)>1.5×ULN;
   Glomerular filtration rate (eGFR) <90 mL/min/1.73 m²;
   Total blood IgG level >ULN.
9. Associated with severe, progressive, or uncontrolled diseases relating to, including but not limited to, the immune system, endocrine system, blood system, urinary system, hepatobiliary system, respiratory system, nervous system, psychiatric system, cardiovascular system, or digestive system, and participation in this study has been determined by the investigator to increase the risk of the subject.
10. Abnormal electrocardiogram with clinical significance, or QTcF>450 ms for male and QTcF>470 ms for female.
11. Hepatitis B surface antigen (HBsAg) positive, hepatitis C antibody (HCV Ab) positive, human immunodeficiency at screening Virus (HIV) antibody positive or serum Treponema pallidum antibody (TP-Ab) positive.
12. Reported history of frequent episodes of orthostatic hypotension within 6 months before screening.
13. Reported history of novel coronavirus infection.
14. Weight change >5% within 6 months prior to screening, or within 1 month prior to screening and planning to use other medications/treatments for weight loss (MAD study only).
15. Reported taking any drug within 14 days before screening or within 5 known half-lives of the first study dose (whichever is longer) any prescription, over-the-counter medicine, vitamin product, or herbal medicine.
16. Participated in other clinical trials and received corresponding research drugs within 3 months before screening.
17. During the test period, there is strenuous exercise, or there is a major change in exercise habits.
18. Reported history of fainting, fainting at sight of blood, or intolerance to venipuncture.
19. Reported history of blood donation within 3 months before screening, or total blood loss >200 ml due to other reasons (except for female menstrual blood loss).
20. Female subjects are breastfeeding during the screening period or during the study, or had a positive serum pregnancy result.
21. Cannot accept a uniform diet and have special requirements for diet.
22. The study researcher determines that the subject is not suitable for participating in this study due to other reasons.

### Concomitant/Prohibited Medication

During the study period (from signing informed consent to the end of the safety follow-up), in principle, no prescription medicines or over-the-counter medicine is allowed. If due to the subject's health condition, the investigator confirms that it is necessary to use any drugs other than the investigational drug, medication or treatment-related information should be recorded in the original study document and electronic case report form (eCRF).

### Primary Endpoints

Safety indicators, including treatment-emergent adverse events (TEAEs), physical examination, vital signs, electrocardiogram, and laboratory test results will be examined to evaluate the safety and tolerability of FP4I in adult subjects.

### Secondary Endpoints

### Single-Dose PK Parameters

Time to maximum plasma concentration (Tₘₐₓ)
Maximum plasma concentration (Cₘₐₓ)
Terminal half-life (t_{1/2z})
Area under the curve from time 0 to infinity (AUC_{0-∞})
Area under the curve from time 0 to t (AUC₀₋ₜ)
Terminal rate constant (λz)

### Steady-State PK Parameters

Steady-state time to reach Cₘₐₓ (T_{max,ss})
Steady-state maximum plasma concentration (C_{max,ss})
Steady-state terminal half-life (t_{1/2,ss})
Area under the curve from time 0 to t at steady-state (AUC_{0-t,ss})
Area under the curve from time 0 to infinity at steady-state (AUC_{0-∞,ss})
Accumulation index Rac: AUC_{0-XX,ss} on day XX/AUC₀₋ₓₓ on day 1
Minimum observed plasma concentration during a dosing interval at steady state (C_{min, ss})

### Immunogenicity Assessment Indicators

Assess the immunogenicity of FP4I in adult subjects by analyzing ADA and NAb levels before and after study administration.

### PD Assessment Metrics (MAD Studies Only)

By analyzing the levels and changes of a series of PD indicators stipulated in the pre- and post-dose regimen of the study, assess FP4I in the PD characteristics of medication in overweight or obese subjects. PD indicators include: (1) Glucose metabolism: fasting blood sugar (FPG), fasting insulin concentration (FBI), fasting C-peptide; (2) Lipid metabolism: total cholesterol (TC), triglyceride (TG),
   low-density lipoprotein cholesterol (LDL-C), high-density lipoprotein cholesterol (HDL-C), Apolipoprotein A (ApoA), Apolipoprotein B (ApoB), Adiponectin. (3) Weight, BMI, waist circumference.

### Statistical Methods

### Analysis Dataset

Full Analysis Set (FAS): will include all randomized groups and those who receive at least one dose of the investigational drug.

Safety Set (SS): will include all patients who have received at least one dose of the investigational drug and have post-medication safety data.

Pharmacokinetics Analysis Set (PKS): will include all subjects receiving at least one dose of the investigational drug with at least one measurable concentration and without protocol deviations that would affect the PK data subject.

Pharmacodynamics Analysis Set (PDS): will include all subjects receiving at least one dose of the investigational drug with at least one pharmacodynamic indicator data, and without protocol deviations that would affect the pharmacodynamic data subjects.

### Statistical Analysis

The statistical analysis plan will specify and describe in detail all the content of the statistical analysis to be carried out based on the main characteristics of the clinical study. Statistical analysis using SAS® 9.4 or above version will be carried out.

### 1. Safety Analysis

Safety analysis is performed based on the safety data sets. Adverse events will be coded using the International Dictionary of Medical Terms (MedDRA). Various adverse events/reactions, including TEAEs, serious adverse events (SAEs)/reactions, important adverse events, and adverse events leading to dropouts are categorized. The laboratory test, vital signs, ECG, and physical examination results will be tabulated.

### 2. Pharmacokinetic Profile Analysis

Pharmacokinetic analysis is performed based on the pharmacokinetic analysis data set. Descriptive statistical analysis is performed on the subjects' drug concentration data. Within each dose group, the mean blood drug concentration-time curve is plotted, and the blood drug concentration-time curve diagram of each subject after drug administration is plotted. The non-compartmental model of the WinNonlin software will be used to calculate the subjects' PK parameters, and perform descriptive statistical analysis on the PK parameters. The linear relationship between the administered dose and PK parameters will be explored.

### 3. Immunogenicity Analysis

The number and percentage of ADA and NAb positive cases are summarized based on the safety analysis set, and descriptive statistical analysis is performed on ADA positive titers to explore the immunogenicity of the drug.

### 4. Analysis of Pharmacodynamic Characteristics

Descriptive statistical analysis of pharmacodynamic indicators is performed based on the pharmacodynamic analysis set.

### 5. Other Analysis

At the conclusion of each dose cohort in the SAD and MAD studies, the SRC will review the safety data and some PK data (if applicable) and decide whether to continue escalation to the next dose cohort or start a new dose cohort trial.

### Study Duration

The SAD phase of this study is up to about 71 days, and the MAD phase is up to about 53 days.

### Example 6: Randomized Double-Blind Phase I Clinical Study of Multi-Dose Escalation

A randomized, double-blind multi-dose escalation phase I clinical trial in adult subjects will be conducted, to evaluate the safety, tolerability, pharmacokinetics, pharmacodynamic and immunogenicity of a recombinant human FGF21-Fc fusion protein (FP4I). The study will enroll patients with dyslipidemia.

The MAD study will include 4 planned groups (i.e., 15 mg QW, 25 mg QW, 25 mg Q2W, and 37.5 mg Q2W) and 2 optional groups (the dosage and administration frequency of the optional groups will be determined upon obtaining data from the 4 planned groups).

This study will enroll subjects with dyslipidemia, 8 subjects in each group, including both male and female. A pilot group of 2 subjects will be tested before the first cohort: one subject receives FP4I and the other one receives placebo randomly. The first cohort will be conducted after approval by the SRC following the safety standard defined in the protocol, i.e., no study drug-related SAE and no greater than grade 3 AE (per CTCAE v.50) after the second dose injection on D15 in the pilot group. The remaining six subjects in cohort 1 (15 mg) will be randomized at 5:1 to receive FP4I or placebo once per week for 4 weeks. The eight subjects in QW group 2 and eight subjects in each Q2W group will be randomized at 6:2 to receive FP4I or placebo. If the subjects in QW group 1 (15 mg) do not experience study drug-related SAE or greater than grade 3 AE on the 7th day after the fourth dose, the studies of cohort 25 mg QW and 25 mg Q2W can be started after SRC review and approval. On day 7 after the 4th dose of 25 mg QW, and after SRC review and approval, the 37.5 mg Q2W study can be started.

The safety assessment and blood sampling for PK, immunogenicity, and PD parameters will be conducted in each study subject according to the study schedule defined in the study protocol. The subjects will stay in a clinical research center on D1 and D20 (or 21) to undertake the multiple blood sampling and will be discharged with the approval of the investigator after completing the safety assessment on an appropriate date. After leaving the research center, subjects are required to return for safety assessment and for PK, immunogenicity, and PD blood sampling according to the study schedule. The subjects will complete the study on D64.

### Study Drug

A recombinant human FGF21-Fc fusion protein for injection (FP4I), 25 mg/vial, will be used in this MAD study. The study drug is refrigerated at 2~8° C., and kept away from light.

### Drug Administration

The study subject will fast for at least 10 hours before study drug administration. Each vial contains 25 mg recombinant FGF21-Fc fusion protein in powder. For injection, the study drug will be reconstituted with distilled water for injection (1.1 mL) to obtain a 25 mg/mL/vial reconstituted solution. The reconstituted drug solution is drawn with a syringe according to the dosage for a particular cohort defined in the protocol. The injection site is at the abdominal wall, with maximal 1.5 mL per injection site

### Patients Inclusion Criteria

Subjects meeting all of the following criteria are eligible for inclusion in this study:
1. Must be able to understand and voluntarily sign written informed consent, including the study drug and collection of all research samples.
2. Male or female aged 18 to 65 years (including the cutoff values).
3. Clinical evidence of TG≥2.0 mmol/L and ≤6.0 mmol/L within 3 months before screening, and fasting TG≥2.0 mmol/L and ≤6.0 mmol/L at screening. Not on lipid-lowering drug treatment or under monotherapy of lipid-lowering drug at the time of screening.
4. Body mass index (BMI): 22~35 kg/m² (including the cutoff values).
5. For subject on anti-diabetic treatment, he/she has to agree to suspend the treatment for at least 10 weeks (starting 7 days before receiving the study treatment till the end of the study).
6. Subjects (including partners) have no plans to become pregnant from two weeks before screening to 3 months after the last study administration, and voluntarily take effective, non-drug contraceptive measures for contraception and have no plans to donate sperm and eggs; or are infertile (received surgical sterilization or are in menopause).

### Exclusion Criteria

Subjects who meet any of the following criteria will not be enrolled in this study:
1. Those who have received any vaccine within 28 days before the first dose of the study drug.
2. Having a history of tumor (whether cured or not, except for basal cell carcinoma and carcinoma in situ).
3. Having a history of allergies, especially allergic to protein drugs or food.
4. Those who smoked more than 5 cigarettes per day within 3 months before screening, or those who could not stop using any tobacco products during the period from 48 hours before the first dose to 48 hours after the last dose.
5. Having a history of alcoholism 3 months before screening (14 units of alcohol per week: 1 unit ≈360 mL of beer or 45 mL of 40% alcohol or 150 mL of wine); or unable to stop taking an alcohol-containing product during the trial, or those who were screened positive for alcohol.
6. Excessive drinking of tea, coffee, or caffeinated beverages (more than 8 cups of corresponding beverages per day; 1 cup is 250 mL) within 3 months before screening, or consumed any caffeinated food or drink (e.g., coffee, strong tea, chocolate, cola, etc.) within 48 hours before the first dose of the study.
7. Having a history of drug abuse within 1 year before screening or have a positive drug screening test during the trial period.
8. Subject with diabetes will be excluded if meeting any of the following conditions
a) Type 1 diabetes, secondary diabetes, or special types of diabetes.
b) Fasting blood glucose >11.1 mg/dL.
c) Evidence or history of diabetic complications with significant end-organ damage, such as proliferative retinopathy and/or macular edema, diabetic nephropathy, diabetic neuropathy.
d) Severe hypoglycemia occurred more than once within 6 months before taking the study drug, or had a history of hypoglycemia but did not know or had insufficient awareness of the symptoms of hypoglycemia.
e) Take two or more anti-diabetic drugs within 4 weeks before screening.
f) On insulin at screening.
g) Known hemoglobinopathies (alpha thalassemia), hemolytic anemia, sickle cell anemia, or hemoglobin <11 g/dL (men) or <10 g/dL (women), or any other disease known to interfere with HbA1c test results.
h) Patients who have received long-term (more than 14 consecutive days) systemic glucocorticoid therapy in the past year (excluding topical, intra-articular and inhaled preparations) or received glucocorticoid therapy within 30 days before screening.
9. Clinically significant abnormal lab results during the screening: i.e. Alanine aminotransferase (ALT), aspartate aminotransferase (AST) or total bilirubin (TBIL)>2.0×ULN; estimated glomerular filtration rate (eGFR)<90 mL/min/1.73 m²; total blood IgG level <LLN; fasting C-peptide <0.8 ng/mL.
10. Having severe, progressive or uncontrolled diseases, including but not limited to, immune system, endocrine system (except T₂DM), hematology system, urinary system, hepatobiliary system, respiratory system, nervous system, mental system, cardiovascular system, digestive system, or having a risk to participate in this study at investigator's discretion.
11. Suffering from uncontrolled hypertension (systolic blood pressure ≥150 mmHg or diastolic blood pressure ≥95 mmHg), with or without stable doses of antihypertensive drugs.
12. Abnormal and clinically significant electrocardiogram, or QTcF>470 ms in males and QTcF>480 ms in females.
13. Hepatitis B surface antigen (HBsAg) positive, hepatitis C antibody (HCV Ab) positive, human immunodeficiency virus (HIV) antibody positive or serum Treponema pallidum specific antibody (TP-Ab) positive at screening.
14. Having a history of frequent episodes of orthostatic hypotension within 6 months before screening.
15. Having a history of pancreatitis or COVID-19 infection.
16. Weight loss >5% within 6 months prior to screening, or have a plan to reduce weight within 1 month prior to screening or during the study period.
17. Participated in other clinical trials and received corresponding research drugs within 3 months before screening.
18. Unable to avoid strenuous exercise or a significant change in exercise and eating habits during the study period.
19. Having a history of fainting spell at sight of needle, haemorrhage, or intolerance to venipuncture.
20. Having a history of blood donation within 3 months before screening, or a history of blood loss more than 300 ml/once (except for female menstrual blood loss).
21. Female subjects were breastfeeding during the screening period or during this study, or had a positive blood pregnancy result.
22. Those who cannot accept a routinely standard diet or have special requirements for diet.
23. The investigator has determined that it is not suitable for the subject to participate in this study for other reasons.

### Combination Therapy

During the study period (from the signing of informed consent to the end of the study), arbitrary addition or reduction of any previously stable prescription or over-the-counter drugs without the investigator's permission is not allowed. From 48 hours before the first/last dose to 48 hours after the first/last dose: no alcohol or alcoholic food; and no caffeinated food or drink (decaffeinated tea and coffee are acceptable). For subjects treated with T₂DM, the antidiabetic drugs will be discontinued from 7 days prior to the administration of the first of study medication until the end of the study (approximately 10 weeks in total). It is not recommended to change the dosage and frequency of concomitant drugs (such as antihypertensive drugs or lipid-lowering drugs) during the study period. The concomitant medication and other treatment-related information will be recorded in the original study file and in the electronic case report form (eCRF).

### Remedial Treatment of Hyperglycemia

Investigators should consider rescue therapy if a subject's fasting plasma glucose is ≥15 mmol/L (270 mg/dl) at 3 consecutive visits. The first choice is to reuse the subject's previous antidiabetic drugs, and the investigators can also use other treatments according to the actual situation. If a life-threatening event occurs, or there is a potential risk of severe complications of hyperglycemia, the investigator may decide to withdraw the subject from the study.

### Primary Endpoints

The safety and tolerability of FP4I are assessed by safety metrics, including treatment-emergent adverse events (TEAEs), physical examination, vital signs, electrocardiogram, and laboratory findings.

### Secondary Endpoints

PK parameters after first dose: Tₘₐₓ, Cₘₐₓ, AUC₀₋₁₆₈, and AUC_{0-∞,ss}
Steady-state PK parameters: T_{max,ss}, C_{max,ss}, C_{min,ss}, t_{1/2,ss}, AUC_{0-t,ss}, AUC₀₋₁₆₈, AUC_{0-∞,ss}, and accumulation index Rac:
AUC₀₋₁₆₈ of last dose/AUC₀₋₁₆₈ of first dose
Cₘₐₓ of last dose/Cₘₐₓ of first dose
Immunogenicity Assessment Indicators: ADA and NAb

### PD Evaluation Parameters

### Lipid Metabolism:

Changes from baseline in triglycerides (TG);
Change from baseline in total cholesterol (TC);
Changes from baseline in low-density lipoprotein cholesterol (LDL-C);
Changes from baseline in high-density lipoprotein cholesterol (HDL-C);
Apolipoprotein A (ApoA) change from baseline;
Apolipoprotein B (ApoB) change from baseline;
Adiponectin change from baseline;

### Glucose Metabolism:

Change from baseline in fasting blood glucose (FPG);
Change from baseline in fasting insulin concentration (Fins);
Change from baseline in fasting C-peptide;
Change from baseline in HbA1c;
Changes in FPG/Fins relative to baseline;
Change from baseline in AUC glucose/AUC insulin;
Change from baseline in HOMA-IR

### Other:

Change in body weight from baseline;
BMI change from baseline;
Change in waist circumference from baseline.

### Statistical Methods

Statistical methods and analysis follow essentially the same methodologies as
described in Example 5.

### Study Duration

The total period of this study is about 78 days (screening period ≤2 w+treatment period 4 w+observation period 5 w).

### Example 7: Randomized Double-Blind Phase I Clinical SAD and MAD Study

A randomized, double-blind, placebo-controlled, sequential parallel group, single and multiple ascending doses (SAD/MAD) study following subcutaneous administration in healthy subjects will be conducted to evaluate the safety, tolerability, pharmacokinetics and immunogenicity potential of FP4I.

### Objectives:

### Primary:

To evaluate the safety and tolerability of FP4I following single and multiple ascending doses by subcutaneous administration.

### Secondary:

To characterize the pharmacokinetics of FP4I following single and multiple ascending doses by subcutaneous administration; and
To determine the immunogenicity potential of FP4I by detecting total anti-drug antibodies (ADA) including neutralizing antibodies.

### Methodology:

Part 1 (SAD): A total of 6 dose cohorts are planned and FP4I dose level of each cohort is determined based on the collective nonclinical data of FP4I (pharmacology and toxicology). The starting dose of 50 mg of FP4I represents about a 75-fold safety margin based on body surface area-adjusted dose at the no-observable-adverse-effect level (NOAEL) dose of 400 mg/kg/day after FP4I was subcutaneously administered to rats twice weekly for 4 weeks and about a 75-fold safety margin based on body surface area-adjusted dose at the NOAEL dose of 200 mg/kg/day after FP4I was subcutaneously administered to monkeys twice weekly for 4 weeks. The starting dose was selected in accordance with scientific standards outlined in the FDA Guidance for Industry, “Estimating the Maximum Safe Starting Dose in Initial Clinical Trials for Therapeutics in Adult Healthy Volunteers” (July 2005).

FP4I exhibited efficacy of anti-hepatic steatosis and lowered nonalcoholic steatohepatitis (NASH) activity score in animal models relevant to NASH. The human equivalent dose of the minimal efficacious dose of 2.5 mg/kg in mice based on body surface area adjustment is about 50 mg with a human body weight of 60 kg. We anticipated that the efficacious dosing regimen of FP4I for the treatment of NASH in human is 100 mg twice weekly and above. The dose level of 6 dose cohorts is 50 mg, 75 mg, 100 mg, 150 mg, 200 mg, and 300 mg, respectively.

A total of 48 subjects will be evaluated with 36 subjects randomized to receive the active drug and 12 subjects randomized to receive placebo in a double-blind fashion (eight subjects per each dose cohort, six subjects randomized to the active drug, and two subjects randomized to placebo). Eligible subjects will be admitted to the clinic on Day −1, one day prior to dosing, remain at the clinic for 3 overnight stays, and be discharged the morning of Day 3, approximately 48 hours post-dose. Subjects will return for outpatient visits on Days 4, 5, 6, 7, 9, 12, and 17 for the collection of the 72, 96, 120, 144, 192, 264, and 384-hour post-dose PK samples. Blood samples for pharmacokinetic (PK) analysis will be collected from Day 1: at pre-dose and 1, 2, 4, 6, 8, 12, 24 (Day 2), 48 (Day 3), 72 (Day 4), 96 (Day 5), 120 (Day 6), 144 (Day 7), 192 (Day 9), 264 (Day 12), and 384 (Day 17) hours post-dose (16 samples each per subject). Blood samples collected from Day 1: at pre-dose and 72 (Day 4), 96 (Day 5), 120 (Day 6), and 144 (Day 7) hours post-dose will also be used for the detection of total anti-drug antibodies (ADA) including neutralizing antibodies. Monitoring of ADA levels will continue for any subject who develops an ADA response until levels return to baseline.

Safety assessments will include monitoring of adverse events (AEs), vital signs (blood pressure, pulse rate, and oral temperature), clinical laboratory findings, 12-lead electrocardiograms (ECGs), physical examination including ophthalmologic exam findings and injection site assessment. Vital sign assessments will be done at screening, on Day −1 (approximately 8 PM), pre-dose and 12, 24, 48, 72, 96, 120, 144, 192, 264, and 384 hours postdose. Clinical labs (including serum IgG) will be performed at screening, on Day −1, Days 3, 7, 9, 12, and 17. Additional assessments of serum IgG levels may be needed until levels return to the normal range, at the discretion of the Investigator. Cortisol levels will be measured in the morning of Day −1 and Day 7. A resting 12-lead ECG will be completed at screening, Day −1, 12, 24, 48, and 384 hours post-dose. Physical exams will be conducted at screening, Day −1, and Day 17.

A follow-up telephone call will be placed to all subjects approximately 5 days after the final outpatient visit to collect adverse event (AE) and concomitant medication information. Single dose escalation will not occur until review of the single dose safety and PK data from the previous dose cohort. After the starting dose of FP4I, dose escalation decisions will be based on safety and tolerability assessments and agreed upon by the Principal Investigator and the Medical Monitor from Ampsource Biopharma Shanghai Inc.

Part 2 (MAD): A total of 24 subjects will receive FP4I (8 subjects in each dose cohort for a total of three cohorts, 6 subjects randomized to active drug, and 2 subjects randomized to placebo per cohort). FP4I dosing regimens (dose levels and duration of administration) will be determined based on the collective SAD PK and safety data from Part 1 and nonclinical data of FP4I (pharmacology and toxicology). Dosing regimen is twice weekly for 2 consecutive weeks (total of 5 doses on Days 1, 4, 8, 11, and 15, respectively). The dose level of the 3 dose cohorts is 50 mg, 100 mg, and 150 mg, respectively. Eligible subjects will be admitted to the clinic on Day −1, one day prior to first dose, remain at the clinic for 17 overnight stays, and be discharged on the morning of Day 17, approximately 48 hours after the final dose. Subjects will return for outpatient visits on Days 18, 19, 20, 21, 23, 26, and 31 for the collection of the 72, 96, 120, 144, 192, 264, and 384-hour post-dose (5th dose) PK samples.

Blood samples for PK analysis will be collected from Day 1: at pre-dose and 1, 2, 4, 6, 8, 12, 24 (Day 2), 48 (Day 3), 72 (Day 4, Pre-2nd dose) hours after the first dose and from Day 15: at pre-dose and 1, 2, 4, 6, 8, 12, 24 (Day 16), 48 (Day 17), 72 (Day 18), 96 (Day 19), 120 (Day 20), 144 (Day 21), 192 (Day 23), 264 (Day 26) and 384 (Day 31) hours post-dose (26 samples each per subject).

Blood samples collected from Day 1: at pre-dose and 72 hours (Day 4, Pre-2nd dose) after the first dose and from Day 15: at 8, 24 (Day 16), 48 (Day 17), 72 (Day 18), 96 (Day 19), 120 (Day 20) and 144 (Day 21) hours post the final dose will also be used for the detection of total anti-drug antibodies (ADA) including neutralizing antibodies. Monitoring of ADA levels will continue for any subject who develops an ADA response until levels return to baseline.

Safety assessments will include monitoring of adverse events (AEs), vital signs (blood pressure, pulse rate, and oral temperature), clinical laboratory findings, 12-lead ECGs, physical examination including ophthalmologic exam findings and injection site assessment. Vital sign assessments will be done at screening, on Day −1 (approximately 8 PM), pre-dose and 12, 24, 48 hours following each dose and at 72 (Day 18), 96 (Day 19), 120 (Day 20), 144 (Day 21), 192 (Day 23), 264 (Day 26) and 384 (Day 31) hours post the final dose. Clinical labs (including serum IgG) will be performed at screening, on Day −1, and 4 hours post dose on Days 1, 4, 8, 11 and 15, and on Days 17, 21, 23, 26 and 31. Additional assessments of serum IgG levels may be needed until levels return to the normal range, at the discretion of the Investigator. Cortisol levels will be measured in the morning of Day −1 and Day 21. A resting 12-lead ECG will be completed at screening, approximately 8 PM on Day −1, and 4 hours post dose on Days 1, 4, 8, 11 and 15, and on Day 31. Physical exams will be conducted at screening, Day −1 and Day 31.

A follow-up telephone call will be placed to all subjects approximately 5 days after the final outpatient visit to collect adverse event (AE) and concomitant medication information. Dose escalation will not occur until review of the safety and PK data from the previous dosed cohorts. Dose escalation decisions will be based on safety, tolerability assessments and agreed upon by the Principal Investigator and the Medical Monitor from Ampsource Biopharma Shanghai Inc.

### Number of Subjects (Planned):

Part 1:48 evaluable subjects, 8 subjects (6 active, 2 placebo) per cohort for a total of six cohorts.

Part 2:24 evaluable subjects, 8 subjects (6 active, 2 placebo) per cohort for a total of three cohorts.

### Selection of Subjects:

### Inclusion Criteria:

1. Are capable of giving informed consent and complying with study procedures;
2. Are between the ages of 18 and 55 years, inclusive;
3. Female subjects have a negative pregnancy test result at screening and admission to the study site, and meet one of the following criteria:
a. Using a medically acceptable form of birth control for at least 1 month prior to screening (3 months on contraceptives) [e.g., hormonal contraceptives (oral subcutaneous, patch, injectable or vaginal ring), implantable device (implantable rod or intrauterine device), or a barrier method];
b. Surgically sterile with documentation for at least 3 months prior to screening by one of the following means:
   Bilateral tubal ligation
   Bilateral salpingectomy (with or without oophorectomy)
   Surgical hysterectomy
   Bilateral oophorectomy (with or without hysterectomy)
c. Postmenopausal, defined as the following:
   Last menstrual period greater than 12 months prior to screening;
   Postmenopausal status confirmed by serum FSH and estradiol levels at screening;
4. Considered healthy by the Investigator, based on subject's reported medical history, full physical examination, clinical laboratory tests, 12-lead ECG, and vital signs;
5. eGFR of 90 mL/min/1.75 m² or greater following review of clinical lab test as determined by the Investigator to be non-clinically significant;
6. Non-smoker and has not been exposed to any products containing nicotine in the last 6 months;
7. Body mass index (BMI) of 18.0 to 32.0 kg/m² inclusive and body weight not less than 50 kg;
8. Willing and able to adhere to study restrictions and to be confined at the clinical research center;
9. Male subjects must agree to utilize a highly effective method of contraception (condom plus spermicide) during heterosexual intercourse from clinic admission until 12 weeks following the end of study visit;
10. Male subjects with female partners of child-bearing potential must agree to use condoms for the duration of the study and until 12 weeks after dosing with the study drug and must refrain from donating sperm for this same period.

### Exclusion Criteria:

1. Clinically significant reported history of gastrointestinal, cardiovascular, musculoskeletal, endocrine, hematologic, psychiatric, renal, hepatic, bronchopulmonary, neurologic, immunologic, lipid metabolism disorders, or drug hypersensitivity as determined by the Investigator;
2. Reported known or suspected malignancy;
3. Reported history of pancreatitis or gall stones;
4. Reported history of unexplained syncope, symptomatic hypotension or hypoglycemia;
5. Reported family history of long QTc syndrome;
6. Reported history of chronic diarrhea, malabsorption, unexplained weight loss, food allergies or intolerance;
7. Poor venous access;
8. Positive blood screen for human immunodeficiency virus (HIV), hepatitis B core (IgG and IgM) and surface antigen (HBsAg), Hepatitis A antibody (IgM), hepatitis C antibody (IgG), or hepatitis E (IgG and IgM) at Screening;
9. Donated or lost >500 mL of blood in the previous 3 months prior to screening;
10. Taken an investigational drug or participated in a clinical trial within 30 days or 5 half lives, whichever is longer prior to first dose of study drug, whichever is longer;
11. Taken any prescription medications (with the exception of hormonal contraceptive) within 14 days or 5 half-lives (whichever is longer) of the first dose of study drug;
12. Reported hospitalization or clinically significant surgery within 6 months prior to screening;
13. A history of prescription drug abuse, or illicit drug use within 9 months prior to screening;
14. A history of alcohol abuse according to medical history (≥2 drinks per day for male and ≥1 drink per day for female) within 9 months prior to screening;
15. A positive screen for alcohol, drugs of abuse at screening or admission;
16. An unwillingness or inability to comply with food and beverage restrictions during study participation;
17. Use of over-the-counter (OTC) medication within 7 days, and/or herbal medications (including St John's Wort, herbal teas, garlic extracts) within 7 days prior to first dose of study drug (Note: Use of acetaminophen at <2 g/day is permitted until 24 hours prior to dosing);
18. Any condition or finding that in the Investigators opinion would put the subject or study conduct at risk if the subject were to participate in the study.

Investigational product, dosage, and mode of administration:

The dosage form for early clinical research is a sterile lyophilized powder for subcutaneous administration and the strength is 25 mg per vial.

### Duration of Study:

Part 1 (SAD): The total duration of participation in the study for each subject is approximately 50 days.
Part 2 (MAD): The total duration of participation in the study for each subject is approximately 70 days.

### Reference Therapy:

Placebo to match FP4I

### Criteria for Evaluation:

### Safety:

Safety assessments will include monitoring of AEs, vital signs (blood pressure, pulse rate, respiratory rate, and oral temperature), clinical laboratory findings including LDL-C, HDL-C, triglycerides, total cholesterol, AST and ALT, 12-lead ECGs, and physical examination, including ophthalmologic exam findings and injection site reaction at various time points during the study.

### Pharmacokinetics:

Part 1: The plasma concentration time data for FP4I will be analyzed using noncompartmental methods. Actual dosing and sampling times will be used for analyses. The primary pharmacokinetics parameters of interest are: Cₘₐₓ, Tₘₐₓ, t_{1/2}, AUCₗₐₛₜ, and AUC_{inf}, MRT, CL/F, Vz/F, Kel for plasma. Additional parameters may be estimated and reported, as appropriate. Actual dosing and sampling times will be used for PK analysis.

Part 2: The following plasma PK parameters for FP4I will be derived using noncompartmental analysis:
For Day 1, Cₘₐₓ, Tₘₐₓ, AUCₗₐₛₜ, and AUCt (t=72 hours).
For Day 15, Cₘₐₓ, Tₘₐₓ, t_{1/2}, and AUCt.

Additional PK information: trough plasma concentration of FP4I, time to reach steady state, as well as the accumulation of FP4I exposure at steady state may be reported. Actual dosing and sampling times will be used for PK analysis.

Immunogenicity: Immunogenicity will be assessed using ADA, including neutralizing antibodies, against FP4I.

### Statistical Methods:

### Safety:

Adverse events will be summarized by system organ class and preferred term; a subject will only be counted once per system organ class and once per preferred term within a treatment. Subject counts and percentages and AE counts will be presented for each treatment and totaled for all treatments. Listings will be presented by subject for all AEs. Clinical laboratory values at each visit and change from baseline will be summarized using descriptive statistics (N, mean, median, standard deviation, minimum, and maximum). All clinical laboratory data will be presented in listings. Within each listing, laboratory values outside the normal ranges will be flagged as either high (H) or low (L). Other safety parameters will be listed and summarized using descriptive statistics. For vital signs and 12-lead ECGs, values outside the normal ranges will be flagged in the listings and change from baseline will be summarized. No formal statistical analysis of safety data is planned.

### Pharmacokinetics:

Individual plasma concentrations and PK parameters of FP4I will be listed and summarized using descriptive statistics. Individual and mean FP4I concentration-time profiles will be presented graphically. No formal statistical analysis is planned.

### Immunogenicity:

Anti-drug antibody and neutralizing antibody results will be listed and the incidence of ADAs and neutralizing antibodies will be summarized using descriptive statistics. No formal statistical analysis of immunogenicity data is planned.

This study will be conducted in accordance with the guidelines of Good Clinical Practices (GCPs) including archiving of essential documents.

One skilled in the art will readily recognize from the disclosure and claims that various changes, modifications, and variations can be made therein without departing from the spirit and scope of the disclosure as defined in the following claims.

What is claimed is:

1. A method of treating a disease or disorder, comprising administering to a subject in need thereof a therapeutically effective amount of an FGF21 fusion protein at least once every four weeks,
wherein the therapeutically effective amount of the FGF21 fusion protein is 5 mg or more,
wherein the FGF21 fusion protein comprises, from the N-terminus to C-terminus, a human fibroblast growth factor 21 or an analog thereof, a flexible peptide linker, at least one rigid unit comprising a carboxyl terminal peptide of human chorionic gonadotropin β subunit, and a fusion partner selected from an immunoglobulin, an Fc fragment thereof, an albumin, and transferrin, and
wherein the disease or disorder is selected from non-alcoholic fatty liver disease (NAFLD), diabetes, diabetic complications, obesity, dyslipidemia, metabolic syndrome, and disorders associated with severe inactivating mutations in the insulin receptor.

2. The method according to claim 1, wherein the human fibroblast growth factor 21 or an analog thereof is selected from polypeptides having an amino acid sequence at least 95% homologous to that of wild-type hFGF21, the flexible peptide linker is selected from L1, L2, L3, L4, and L5, the at least one rigid unit is selected from CTP1, CTP2, CTP3, CTP4, and CTP5, and the fusion partner is selected from vFcγ1, vFcγ2-1, vFcγ2-2, vFcγ2-3, and vFcγ4.

3. The method according to claim 1, wherein the FGF21 fusion protein is selected from FP4A, FP4B, FP4C, FP4D, FP4E, FP4F, FP4G, FP4H, and FP4I.

4. The method according to claim 1, wherein the FGF21 fusion protein comprises an amino acid sequence of SEQ ID NO: 8.

5. The method according to claim 1, wherein the FGF21 fusion protein is a protein encoded by a nucleotide sequence of SEQ ID NO: 9.

6. The method according to claim 1, wherein the therapeutically effective amount of the FGF21 fusion protein is from 5 mg to 400 mg; preferably, the therapeutically effective amount of the FGF21 fusion protein is from 10 mg to 300 mg;
preferably, the therapeutically effective amount of the FGF21 fusion protein is from 15 mg to 60 mg.

7.-8. (canceled)

9. The method according to claim 1, wherein the therapeutically effective amount of the FGF21 fusion protein is 10 mg or more.

10. The method according to claim 1, wherein the therapeutically effective amount of the FGF21 fusion protein is 15 mg or more;
preferably, the therapeutically effective amount of the FGF21 fusion protein is 20 mg or more;
preferably, the therapeutically effective amount of the FGF21 fusion protein is 25 mg or more;
preferably, the therapeutically effective amount of the FGF21 fusion protein is 30 mg or more;
preferably, the therapeutically effective amount of the FGF21 fusion protein is 35 mg or more;
preferably, the therapeutically effective amount of the FGF21 fusion protein is 37.5 mg or more;
preferably, the therapeutically effective amount of the FGF21 fusion protein is from 15 mg to 37.5 mg.

11.-16. (canceled)

17. The method according to claim 1, wherein the therapeutically effective amount of the FGF21 fusion protein is administered at least once every three weeks, at least once every two weeks, or at least once every week.

18. The method according to claim 1, wherein the therapeutically effective amount of the FGF21 fusion protein is administered at least twice a week;
preferably, the therapeutic effective amount of FGF21 fusion protein is administered three times a week, four times a week, five times a week, six times a week, or daily.

19. (canceled)

20. The method according to claim 1, wherein the therapeutically effective amount of the FGF21 fusion protein is selected from 25 mg, 50 mg, 100 mg, 150 mg, and 200 mg;
preferably, the therapeutically effective amount of the FGF21 fusion protein is selected from 50 mg, 75 mg, 100 mg, 150 mg, 200 mg, and 300 mg;
preferably, the therapeutically effective amount of the FGF21 fusion protein is selected from 75 mg, 100 mg, and 150 mg, and wherein the therapeutically effective amount of the FGF21 fusion protein is administered twice a week.

21.-22. (canceled)

23. The method according to claim 1, wherein the therapeutically effective amount of the FGF21 fusion protein is 15 mg administered once a week; the therapeutically effective amount of the FGF21 fusion protein is 25 mg administered once a week; the therapeutically effective amount of the FGF21 fusion protein is 25 mg administered once every two weeks; or the therapeutically effective amount of the FGF21 fusion protein is 37.5 mg administered once every two weeks.

24. The method according to claim 1, wherein the therapeutically effective amount of the FGF21 fusion protein is selected from 50 mg, 100 mg, and 150 mg, and wherein the therapeutically effective amount of the FGF21 fusion protein is administered twice a week.

25. The method according to claim 1, wherein the FGF21 fusion protein is administrated by parenteral injection;
preferably, the FGF21 fusion protein is administrated by subcutaneous or intravenous injection.

26. (canceled)

27. The method according to claim 25, wherein the FGF21 fusion protein is administrated by subcutaneous injection and the injection site is at the abdominal wall, with <1.5 mL per injection point.

28. The method according to claim 1, wherein the therapeutically effective amount of the FGF21 fusion protein is administered for a total of at least one time.

29. The method according to claim 1, wherein the therapeutically effective amount of the FGF21 fusion protein is administered for a total of at least 5 times;
preferably, the therapeutically effective amount of the FGF21 fusion protein is administered for a total of at least 8 times.

30. (canceled)

31. The method according to claim 1, wherein a pharmaceutical composition comprising the FGF21 fusion protein and at least one pharmaceutically acceptable carrier is administered.

32. (canceled)

33. The method according to claim 1, wherein the disease or disorder is NAFLD;
preferably, the disease or disorder is NASH;
preferably, the disease or disorder is dyslipidemia.

34.-37. (canceled)
